(19) United States
(12) Patent Application Publication
(10) Pub. No.: US 2026/0026729 A1
(43) Pub. Date: Jan. 29, 2026

(54) SYSTEM AND METHOD FOR A BISPECTRAL INDEX SENSOR

(71) Applicant: Covidien LP, Mansfield, MA (US)

(72) Inventors: Shy Hemed, Jerusalem (IL); Ilan Breskin, Tel Aviv-Yafo (IL); Rachel Gabriel, Tel Aviv (IL); Yedidia Blonder, Jerusalem (IL); Denis Glozman, Modiin (IL); Alexander Rabkin, Mevaseret Zion (IL); Shai Fleischer, Modiin (IL)

(73) Assignee: Covidien LP, Mansfield, MA (US)

(21) Appl. No.: 19/253,432

(22) Filed: Jun. 27, 2025

Related U.S. Application Data
(60) Provisional application No. 63/675,560, filed on Jul. 25, 2024.

(51) Int. Cl. A61B 5/291; A61B 5/00; A61B 5/257; A61B 5/296; A61B 5/372; A61B 5/395

(52) U.S. Cl. CPC A61B 5/291; A61B 5/257; A61B 5/296; A61B 5/372; A61B 5/395; A61B 5/4821; A61B 2562/043; A61B 2562/164

(57) ABSTRACT

A sensor may concurrently measure hypnotic condition of a patient and a paralytic condition of the patient. The sensor may include a set of electrodes configured to couple to a patient via respective adhesive electrode assemblies, where each electrode of the set of electrodes is configured to measure an EEG signal or an EMG signal and a connector configured to couple the sensor to a patient monitoring device. The sensor may also include a tail component coupled to an electrode assembly of the first electrode of the set of electrodes and the connector, where the tail component is non-adhesive and a sticker positioned between the tail component and the connector, wherein the sticker is configured to adhere to the patient or the sensor to adjust a position of the tail component, the connector, or both.

### CROSS-REFERENCE TO RELATED APPLICATION(S)

This application claims the benefit of U.S. Provisional Application No. 63/675,560, filed Jul. 25, 2024, which is incorporated herein by reference in its entirety.

### TECHNICAL FIELD

The present disclosure relates generally to medical devices and, more particularly, to sensors used for sensing brain activity and other physiological parameters of a patient.

### BACKGROUND

Patient monitoring devices, such as brain monitoring devices (e.g., electroencephalogram (EEG) monitoring devices, electromyography (EMG) monitoring devices, and other brain monitoring devices), may be configured to monitor various physiological parameters of a patient. For example, sensors used for brain monitoring may include one or more electrodes for placement on the forehead to noninvasively acquire an EEG and/or EMG signal. Proper placement of the electrode(s) of the sensor on the forehead helps to correctly locate physiologically relevant signals of the patient and calculate the desired physiological parameters such as the patient's depth of consciousness, level of sedation, paralytic state, and other parameters. These parameters are monitored during medical procedures involving anesthesia in order to keep the level of sedation within a desired range, to reduce complications or adverse events during the procedure or during recovery. Misplacement of the electrodes may increase algorithmic work, filtering, and activating to obtain the physiological parameters of the patient, which may result in potentially misreporting the physiological parameters.

### SUMMARY

Certain embodiments commensurate in scope with the originally claimed subject matter are summarized below. These embodiments are not intended to limit the scope of the disclosure. Indeed, the present disclosure may encompass a variety of forms that may be similar to or different from the embodiments set forth below.

In one embodiment, a sensor may include a set of electrodes configured to couple to a patient via respective adhesive electrode assemblies, where each electrode of the set of electrodes is configured to measure an EEG signal or an EMG signal and a connector configured to couple the sensor to a patient monitoring device. The sensor may also include a tail component coupled to an electrode assembly of the first electrode of the set of electrodes and the connector, where the tail component is non-adhesive and a sticker positioned between the tail component and the connector, wherein the sticker is configured to adhere to the patient or the sensor to adjust a position of the tail component, the connector, or both.

In another embodiment, a system may include a set of electrode assemblies configured to couple to a patient, wherein each electrode of the set of electrode assemblies is configured to measure an EEG signal or an EMG signal, a transparent layer coupled to each electrode assembly of the set of electrode assemblies, and a tail component coupled to a first electrode assembly of the set of electrode assembly and a connector.

Various refinements and features noted above may exist in relation to various aspects of the present disclosure. Further features may also be incorporated in these various aspects as well. These refinements and additional features may exist individually or in any combination. For instance, various features discussed below in relation to one or more of the illustrated embodiments may be incorporated into any of the above-described aspects of the present disclosure alone or in any combination. The brief summary presented above is intended only to familiarize the reader with certain aspects and context of embodiments of the present disclosure without limitation to the claimed subject matter.

### BRIEF DESCRIPTION OF THE DRAWINGS

Advantages of the disclosed techniques may become apparent upon reading the following detailed description and upon reference to the drawings in which:

FIG. 1 is a schematic diagram of an embodiment of a patient monitoring system including a patient monitor, in accordance with an aspect of the present disclosure;

FIG. 2A is a perspective view of an embodiment of a sensor of the patient monitoring system of FIG. 1, in accordance with an aspect of the present disclosure;

FIG. 2B is a top view of another embodiment of the sensor of the patient monitoring system of FIG. 1, in accordance with an aspect of the present disclosure;

FIG. 2C is a top perspective view of a combined NMT and EEG sensor, in accordance with an aspect of the present disclosure;

FIG. 2D is a lower perspective view of the sensor of FIG. 2C;

FIG. 2E is a front view of a combined NMT and EEG sensor applied to a patient, according to an aspect of the present disclosure;

FIG. 2F is a top view of a bilateral combined NMT and EEG sensor, according to an aspect of the present disclosure;

FIG. 2G is a graph of three NMT responses in a subject with a reference electrode and stimulating electrodes applied to the same hemisphere;

FIG. 2H is a graph of two NMT responses in a subject with a reference electrode and stimulating electrodes applied to opposite hemispheres;

FIG. 3A is a perspective view of the sensor of FIG. 2A applied to a patient of the patient monitoring system of FIG. 1, in accordance with an aspect of the present disclosure;

FIG. 3B is a perspective view of the sensor of FIG. 2A applied to a patient of the patient monitoring system of FIG. 1, in accordance with an aspect of the present disclosure;

FIG. 3C is a perspective view of the sensor of FIG. 2A applied to a patient of the patient monitoring system of FIG. 1, in accordance with an aspect of the present disclosure;

FIG. 3D is a perspective view of the sensor of FIG. 2A applied to a patient of the patient monitoring system of FIG. 1, in accordance with an aspect of the present disclosure;

FIG. 4A is a top view of another embodiment of the sensor of the patient monitoring system of FIG. 1, in accordance with an aspect of the present disclosure;

FIG. 4B is a top view of another embodiment of the sensor of the patient monitoring system of FIG. 1, in accordance with an aspect of the present disclosure;

FIG. 5A is a top-down view of the sensor of FIG. 4B applied to a patient of the patient monitoring system of FIG. 1, in accordance with an aspect of the present disclosure;

FIG. 5B is a side view of the sensor of FIG. 4B applied to a patient of the patient monitoring system of FIG. 1, in accordance with an aspect of the present disclosure;

FIG. 5C is a close-up side view of the sensor of FIG. 4B applied to a patient of the patient monitoring system of FIG. 1, in accordance with an aspect of the present disclosure;

FIG. 6 is a top view of the sensor of FIG. 4B with a color indicator, in accordance with an aspect of the present disclosure;

FIG. 7 is a top view of another embodiment of the sensor of FIG. 4B of the patient monitoring system of FIG. 1, in accordance with an aspect of the present disclosure;

FIG. 8 is a front view of aspects of the sensor of FIG. 7 applied to a patient of the patient monitoring system of FIG. 1, in accordance with an aspect of the present disclosure;

FIG. 9 is a top view of another embodiment of the sensor of the patient monitoring system of FIG. 1, in accordance with an aspect of the present disclosure;

FIG. 10 is a schematic view of another embodiment of the sensor of the patient monitoring system of FIG. 1, in accordance with an aspect of the present disclosure;

FIG. 11 is a top view of another embodiment of the sensor of the patient monitoring system of FIG. 1, in accordance with an aspect of the present disclosure;

FIG. 12 is a close up view of the embodiment of the sensor of FIG. 11 of the patient monitoring system of FIG. 1, in accordance with an aspect of the present disclosure;

FIG. 13 is a bottom view of another embodiment of the sensor of the patient monitoring system of FIG. 1, in accordance with an aspect of the present disclosure;

FIG. 14 is a block diagram of the patient monitoring system of FIG. 1, in accordance with an aspect of the present disclosure; and

FIG. 15 is a front view of a graphical user interface (GUI) displayed on a user interface of a patient monitoring device of the patient monitoring system of FIG. 1, in accordance with an aspect of the present disclosure.

FIG. 16 is a flowchart of a method of operating a combined NMT and EEG sensor, according to an aspect of the present disclosure.

### DETAILED DESCRIPTION

One or more specific embodiments of the present techniques will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

When introducing elements of various embodiments of the present disclosure, the articles “a,” “an,” and “the” are intended to mean that there are one or more of the elements. The terms “comprising,” “including,” and “having” are intended to be inclusive and mean that there may be additional elements other than the listed elements. Additionally, it should be understood that references to “one embodiment” or “an embodiment” of the present disclosure are not intended to be interpreted as excluding the existence of additional embodiments that also incorporate the recited features.

Aspects of the disclosure present brain monitoring sensors with features that improve clinical workflow, patient comfort, and physiologic parameter monitoring. In various examples described below, a brain monitoring sensor includes sensing, stimulating, and reference electrodes that can be used in combination for monitoring both EEG (electroencephalography) and NMT (neuro-muscular transmission) parameters. Combining EEG and NMT monitoring in one sensor requires co-use of the sensor electrodes. Various examples below present layouts and configurations of electrodes that enable this combined sensing. As a result, a single sensor can provide both EEG and NMT signals. The EEG signal is processed to determine the patient's brain activity, which indicates the depth of anesthesia. In one embodiment the NMT monitoring uses an evoked electromyogram (EMG) signal to determine the patient's muscle response, which indicates the patient's paralytic state. Providing both of these signals from a single sensor enables medical professionals to better understand the patient's overall depth of consciousness and manage the administration of anesthesia appropriately and safely. Additionally, examples of sensors herein include indicators, accessories, and flexibility that improve patient comfort and clinical workflow.

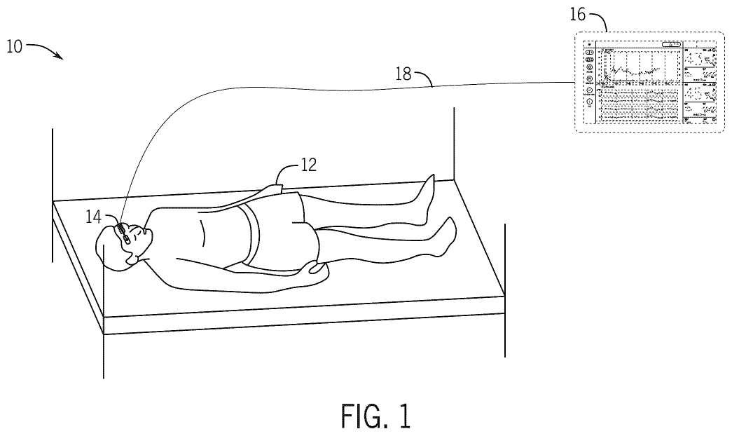

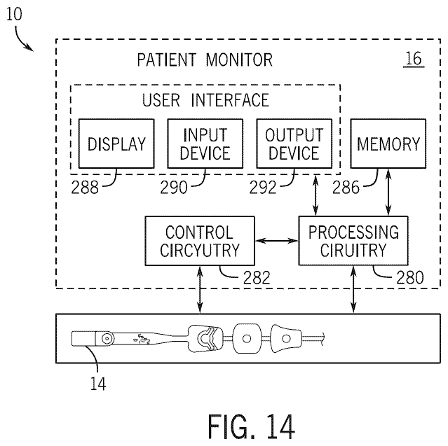

A clinician (e.g., nurse, doctor, technician) may desire to monitor physiological parameters of a patient, such as during anesthesia or other sedation. By way of example, FIG. 1 illustrates a patient monitoring system 10 with a patient 12 and a sensor (e.g., an anesthesia monitoring sensor) 14 configured to monitor one or more physiological parameters of the patient 12. In an embodiment, the sensor 14 includes electrodes that measure electrical activity of the patient's brain and muscles, for the purposes of measuring a patient's depth of consciousness during procedural sedation. The electrodes detect electrical activity such as a patient's EEG, spontaneous electromyogram (EMG), and/or evoked EMG for neuromuscular transmission monitoring (NMT signal). An example depth of consciousness monitor is the BIS™ bispectral index monitor from Medtronic, which measures a level of consciousness by algorithmic analysis of a patient's electroencephalography (EEG) during general anesthesia. The sensor 14 may also include any suitable sensor (e.g., EEG sensors, electromyography (EMG) sensors, other brain monitoring devices) configured to monitor physiological parameters of the patient 12 during a surgical procedure. The sensor 14 may be coupled to a monitor 16 and configured to transmit data (e.g., raw or processed sensor data, measured physiological parameters) to the monitor (e.g., EEG monitor, EMG monitor) 16 for processing. For NMT monitoring, the monitor 16 also sends a signal to the sensor to deliver the stimulating pulses, as described further below. As illustrated, the sensor 14 is physically coupled to the patient monitor 16 by a cable 18. In other instances, the monitor 16 and the sensor 14 may be wirelessly coupled, such as via a network, Bluetooth, Sidelink, or the like. As further described with respect to FIG. 14, the monitor 16 may include processing circuitry that receives, filters, and/or processes the sensor data from the sensor 14. The monitor 16 may also include a user interface and/or a display to display the physiological parameters for the clinician to view. As such, the clinician may monitor the physiological parameters of the patient 12.

The clinician may couple the sensor 14 to the patient 12 to measure the physiological parameters of the patient 12. The coupling may be a direct placement of the sensor 14 on the patient's skin. In an embodiment, the sensor 14 is reversibly attached to the patient via a biocompatible adhesive. The sensor 14 may include a single strip with one or more electrodes for placement on a temple and/or a forehead of the patient 12 to non-invasively acquire an EEG, EMG, and/or NMT signal and to deliver stimulating pulses. Proper placement of the sensor 14 and/or the electrodes may facilitate accurate calculations of the physiological parameters (e.g., BIS™ bispectral index, paralytic state, spontaneous EMG, others), while misplacement of the electrodes may increase algorithmic work, filtering, and artifact removal to obtain the physiological parameters, which may result in potentially misreporting the physiological parameters.

In certain instances, multiple sensors measuring different parameters are placed on a patient during a procedure, such as an EEG sensor as well as an oximetry sensor (e.g., a pulse oximetry sensor or regional oximetry sensor applied to the forehead). The shape and/or size of the various sensors relative to the anatomy of the individual patient may pose challenges for proper placement of the sensors. For example, the forehead of the patient 12 may be small in comparison to the desired sensor or sensors, which may make proper placement difficult. Trimming the sensors to reduce their size to fit onto a patient is not a preferred practice, as trimming may involve cutting away adhesive regions, which may reduce the available surface area adhered to the forehead of the patient 12 and in turn may reduce the overall strength of adhesive attachment to the patient. Trimming may then result in the use of additional adhesives or accessories (e.g., gauze, gel) to hold the sensors in place. The use of additional adhesives may cause pressure sores on the patient 12 and/or markings on the skin when removing the sensors, such as after surgery and/or monitoring. Additionally or alternatively, these various workflows may cause portions of the sensors to move, twist, or shear against the patient's skin during the procedure.

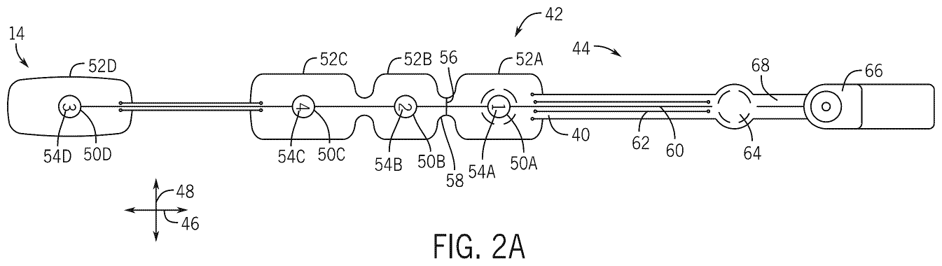

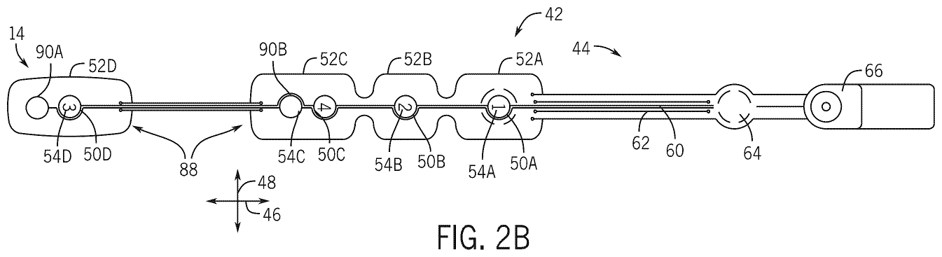

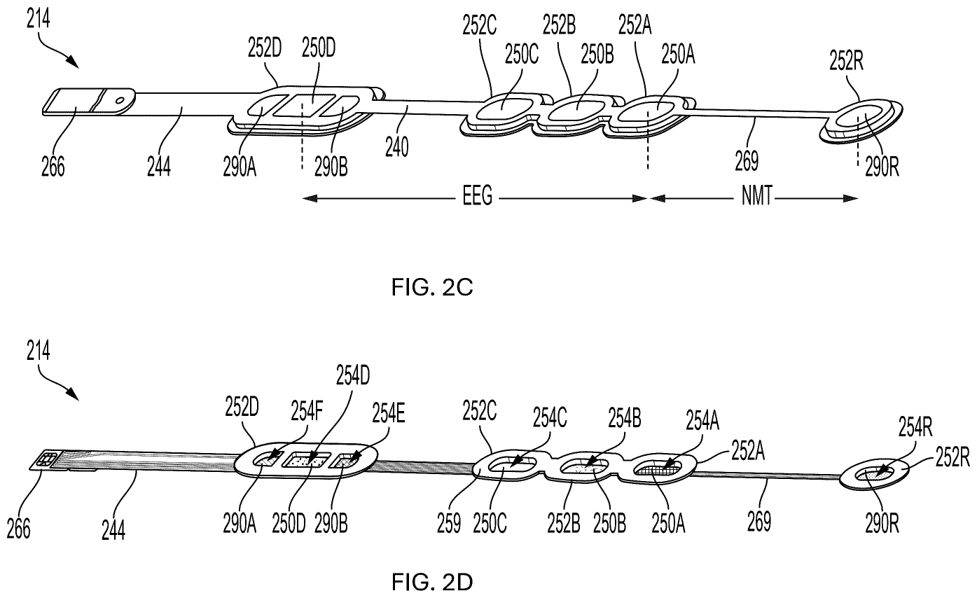

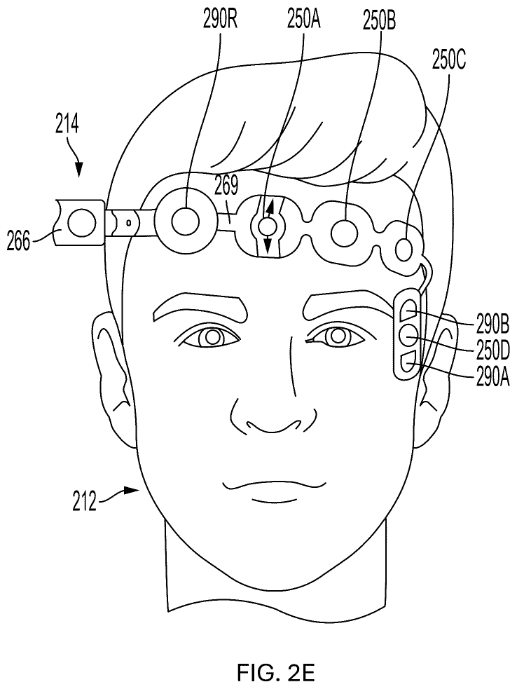

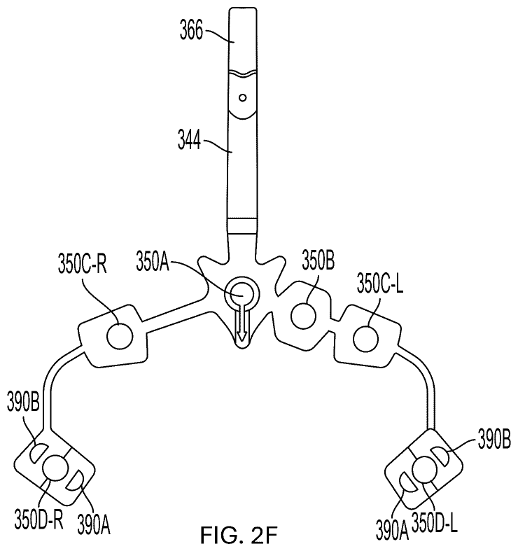

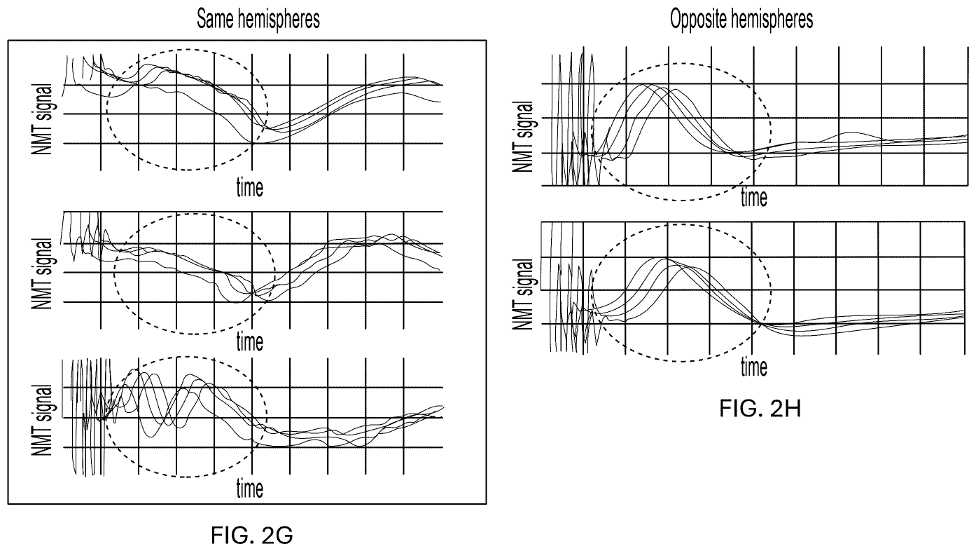

With the foregoing in mind, the present disclosure is generally directed to sensor designs and/or shapes for the sensor 14 to enable proper placement of the sensor 14 on the patient 12. For example, FIGS. 2A-F, 3A-D, 4A, 4B, 5A-C, 6, 7, and 9-13 illustrate embodiments of the sensor with improved form factor and/or adhesion to reduce or eliminate skin markings, reduce sensor footprint (e.g., shape and/or size), and/or simplify a number of steps (e.g., workflow) performed by the clinician. As discussed herein, the sensor designs and/or shapes are configured to help in the placement of the sensor and to facilitate proper positioning of the sensor. In this manner, the sensor 14 may be properly adhered to the patient's temple and/or forehead to facilitate accurate obtainment of physiological data, such as an EEG signal for depth of consciousness monitoring, a spontaneous EMG signal for motor movement monitoring, and/or an evoked EMG for neuromuscular transmission (NMT) monitoring. In certain instances, the sensor 14 may concurrently measure the hypnotic condition and the paralytic condition of the patient 12 and transmit the information for display on the monitor 16. The clinician may view and take the information into account when administering anesthesia to the patient and/or managing the patient's recovery from anesthesia.

FIG. 2A is a perspective view of an embodiment of the sensor 14 of the patient monitoring system 10, in accordance with an aspect of the present disclosure. The sensor 14 may include a substrate 40 (e.g., clongated substrate) with a body component 42 and a tail component 44. The body component 42 and the tail component 44 may be positioned along a longitudinal axis 46 of the sensor 14 (horizontal in the orientation of FIG. 2A). The sensor 14 may include a width extending perpendicularly from the longitudinal axis 46 and along a transverse axis 48 (vertical in the orientation of FIG. 2A). The body component 42 extends a greater distance in the transverse direction than does the tail component 44.

The body component 42 may include a first electrode 50A, a second electrode 50B, a third electrode 50C, and a fourth electrode 50D (collectively referred to herein as electrodes 50 or a set 50 of electrodes). In an embodiment, the electrodes 50 are sensing electrodes that sense and detect electrical signals from the patient, such as EEG signals and/or spontaneous EMG signals, which may be referred to as the sensor signals. The sensor 14 transmits the sensor signals to the monitor 16 for processing into desired physiologic parameters. In an embodiment, each electrode of the set 50 of electrodes is seated within a respective portion of the body component 42 to form an electrode assembly. The body component 42 may include a first electrode assembly 52A carrying the first electrode 50A, a second electrode assembly 52B carrying the second electrode 50B, a third electrode assembly 52C carrying the third electrode 50C, and a fourth electrode assembly 52D carrying the fourth electrode 50D (collectively referred to herein as “electrode assembly 52”). At each electrode assembly 52, the substrate of the body component 42 supports the respective electrode 50 in a direction along the longitudinal axis 46 and the transverse axis 48. This design counteracts peeling forces and/or reduces adhesion shear by distributing load in different directions, such as outwards from the electrode 50. In an embodiment, each electrode assembly 52 extends a greater distance in the longitudinal direction 46 than it does in the transverse direction 48, thus distributing loads along the longitudinal axis 46 of the sensor 14 and reducing the footprint of the sensor in the transverse direction 48. By reducing a size of the electrode assemblies 52, the sensor 14 may occupy less space on the temple and/or forchead of the patient 12 and allow for other sensors and/or technology to adhere to the temple and/or forehead of the patient 12. In addition, the clinician may attach the sensor 14 without cutting the sensor 14, thereby reducing a number of steps performed. Although the illustrated sensor 14 of FIG. 2A includes four electrodes 50 and four electrode assemblies 52, the sensor 14 may include any suitable number of electrodes 50 and electrode assemblies 52 for monitoring physiological parameters of the patient 12.

In certain instances, a design and/or shape of the fourth electrode assembly 52D may improve adhesion of the sensor 14 to the patient 12. For example, a length of the fourth electrode assembly 52D along its longest dimension is longer than a longest dimension of the other electrode assemblies 52. This extended length of electrode assembly 52D provides additional interfacing (e.g., surface area) between the fourth electrode assembly 52D and the skin of the patient 12. The additional interfacing may improve adhesion of the fourth electrode assembly 52D. Each electrode assembly 52 may be characterized by a total adhesive surface area. In an embodiment, the fourth electrode assembly 52D may have a greater total adhesive surface area relative to the other electrode assemblies 52. In an embodiment, the total adhesive surface area of electrode 52D is at least 10% greater than that of each other electrode assembly 52.

The sensor 14 may include one or more gel pockets 54 configured to hold conductive gel. The conductive gel may be configured to adhere the sensor 14 to the temple and/or forehead of the patient 12 and/or conduct electrical signals between the electrodes 50 and the patient's skin. As illustrated, the sensor 14 may include a first gel pocket 54A, a second gel pocket 54B, a third gel pocket 54C, and a fourth gel pocket 54D (collectively referred to herein as “gel pocket 54”) that align with the first electrode 50A, the second electrode 50B, the third electrode 50C, and the fourth electrode 50D, respectively. The gel pockets 54 may be any suitable shape or size configured to hold an amount of conductive gel. In an embodiment, the gel pockets 54 are rectangular in shape, which aligns with the reduced footprint of the electrode assemblies 52. For example, a shape of the gel pockets 54 may be configured to reduce or prevent gel leakage from the gel pockets and/or improve adherence of the sensor 14 to the patient 12. The gel may be conductive (conducting electrical signals from the skin to the electrode) and/or adhesive (improving adhesion of the sensor 14 to the patient 12). As such, the electrodes 50 may be adhered to the temple and/or forehead of the patient 12 via the conductive gel from the gel pockets 454.

In use, the electrodes 50 sense electrical signals from the tissue below each respective electrode, and the signals are combined to produce an EEG signal of the patient. The first electrode 50A is the EEG reference electrode, which is positioned at the center of the patient's head (above the nose) and provides a baseline or reference for the measured signals. The second electrode 50B is the patient ground electrode. The third electrode 50C is an EMG artifact reduction electrode. The fourth electrode 50D is the EEG measurement electrode. Together, these electrodes produce a measurable EEG signal that can be further processed to determine brain activity and/or depth of consciousness metrics for the patient.

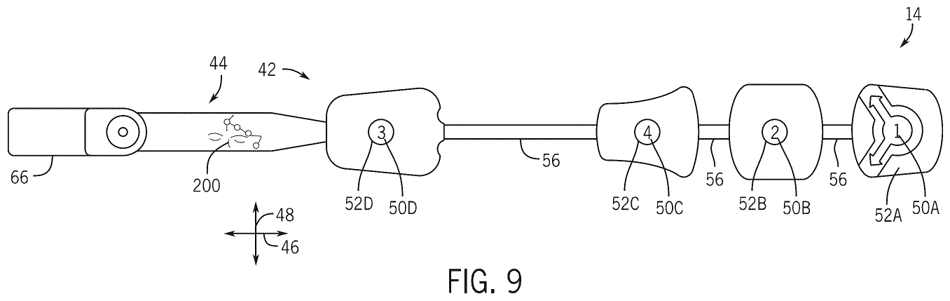

The body component 42 may include a bridge 56 between consecutive electrode assemblies 52 to help fix a distance between the electrodes 50 and reduce lateral movement of the electrodes 50 along the longitudinal axis 46 of the sensor 14 during placement of the sensor 14 onto the patient 12. The bridge 56 may include a curved contour 58 to reinforce proper placement of the sensor 14, such as by facilitating the angular placement of the sensor 14. As illustrated, the curvature 58 may be of a concave nature configured to trace up and around the lateral and top edges of the patient's eyebrow to reinforce correct placement of the electrodes 50. In certain instances, the sensor 14 may include elastic bridges 56 to improve ease of positioning the electrodes 50. As further discussed with respect to FIG. 9, the bridges 56 between each electrode 50 may be flexible.

The tail component 44 of the sensor 14 may include a tail section 60 with a longitudinal slit 62, a sticker 64, and a connector 66 (e.g., a cable connector) at the end of the tail opposite the electrodes 50. The connector 66 connects to the cable 18 (see FIG. 1) that connects to the monitor 16. The tail component 44 may carry electrical connectors on or in the tail component that electrically couple the electrodes 50 to the cable 18. The tail component 44 may be coupled to the first electrode assembly 52A at a first end and the cable 18 at a second end. In an embodiment, the tail component 44 is intermittently adhesive, with the sticker 64 providing an adhesive region or spot adhesion between a non-adhesive tail section 60 and a non-adhesive additional section 68 connecting to the connector 66. In the illustrated embodiment, the sensor 14 is laid flat. However, by bending and/or twisting the tail component 44, the sticker 64 can be moved relative to the electrodes 50 and adhered on the patient and/or directly on the sensor 14 to provide different sensor configurations. In this manner, the tail component 44 can maintain electrical connectivity between the cable 18 and the electrodes 50 while also being folded or bent as desired.

The connector 66 may be positioned at the second end of the tail 44 and may include a plug or connector configured to couple (e.g., reversibly couple) to the cable 18 to transmit physiological parameters from the sensor 14 to the patient monitor 16. The connector 66 and/or the cable 18 may be heavy components in comparison to the body component 42, and therefore movement of the connector 66 and/or the cable 18 may apply pressure (e.g., adhesion shear) to the patient 12 and/or the electrode assemblies 52. To reduce the twisting and pressure action caused by the connector 66 and/or the cable 18 on the electrodes, the tail component 44 may include the tail section 60 with the slit 62. For example, a portion of the tail section 60 may be removed or cut to create the slit 62. The slit 62 may extend all or part of the distance from the first electrode assembly 52A to the sticker 64 along the longitudinal axis 46 of the sensor 14. The slit 62 may separate the tail section 60 into a first portion and a second portion, which may improve flexibility of the tail section 60 and reduce pressure (e.g., adhesion shear) caused by movement of the tail section 60. Although the illustrated sensor 14 of FIG. 2A includes two slits 62, the sensor 14 may include any suitable number of slits 62 along the tail section 60 or the additional section 68. The slit 62 may be positioned to avoid internal electrical conductors/traces and/or to permit the electrical conductors/traces to be uninterrupted between the electrodes 50 and the cable 18.

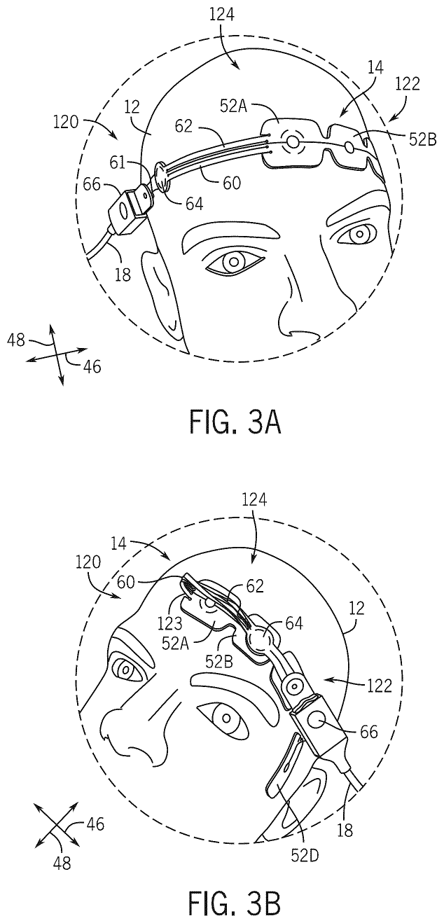

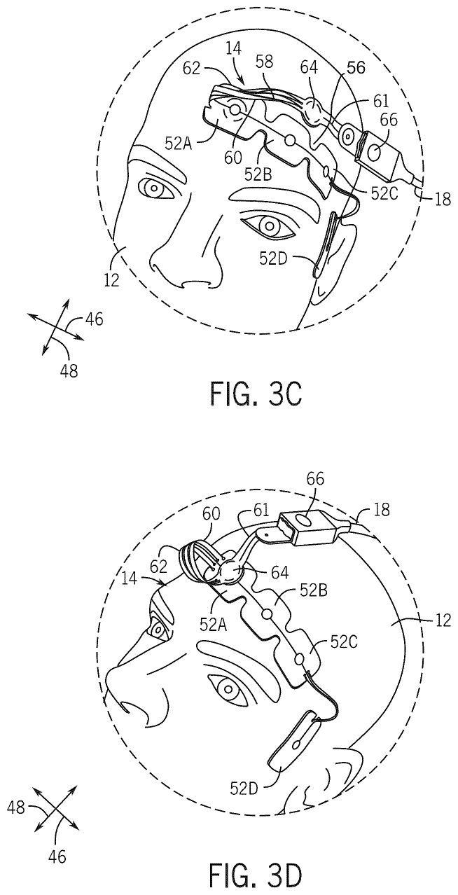

The sticker 64 may provide an adhesive region positioned between two non-adhesive regions 60, 68. The sticker 64 may also reduce and/or eliminate pressure caused by the connector 66 and/or the cable 18. The sticker 64 may be configured to adhere to the patient 12 and/or to an electrode assembly 52 of the sensor 14 and absorb pressure exerted by the connector 66 and/or the cable 18. As illustrated, the sticker 64 may be positioned between the tail section 60 and the additional tail section 68. The additional tail section 68 may be shorter than the tail section 60. Due to the relatively shorter length of the additional tail section 68, movement of the connector 66 may be limited, thereby reducing an amount of pressure exerted by the connector 66 and/or the cable 18 onto the sticker 64. In use, the sticker 64 may be used to secure the tail section 60 in different positions to reduce movement of the connector 66 and/or the cable 18, thereby reducing pressure from the tail section 60 on the patient's skin. As further illustrated in FIGS. 3A-D, the sticker 64 may adhere to different parts of the temple and/or forehead of the patient 12 or to the electrode assemblies 52 of the sensor 14. For example, the placement of the sticker 64 may cause the tail section 60 to rotate in a direction away from the temple and/or forehead of the patient 12, which may also reduce interference of the cable 18 during patient monitoring. In another example, the sticker 64 may adhere to an electrode assembly 52 to cause the tail section 60 to fold over one or more electrode assemblies 52, thereby reducing the sensor footprint on the temple and/or forehead of the patient 12. As such, the sensor 14 may occupy less space on the temple and/or forehead of the patient 12, improve adhesion, and/or reduce markings on the patient's skin.

FIG. 2B is a top view of an embodiment of the sensor 14 of the patient monitoring system 10, in accordance with an aspect of the present disclosure. The sensor 14 of FIG. 2B is substantially similar to the sensor 14 of FIG. 2A except the sensor 14 of FIG. 2B includes a neuromuscular transmission (NMT) unit 88 with a first NMT electrode 90A and a second NMT electrode 90B (collectively referred to herein as “NMT electrodes 90”). The NMT unit 88 may be integrated into the sensor 14 to measure neuromuscular contractions of the patient during anesthesia monitoring. For example, the NMT unit 88 include a pair of stimulating electrodes that deliver electric pulses to stimulate nerves of the patient 12 beneath the stimulating electrodes. When the nerve responses of the patient are intact, the nerves cause the muscles of the patient 12 to contract in response to the electric pulses. This contraction is measured by recording electrodes. When the nerve responses of the patient are blocked (such as from a paralytic agent), the nerves do not respond, and the corresponding muscles do not contract. This is also measured by the recording electrodes. The NMT unit 88 may also include a pair of recording electrodes to measure the electrical activity of the muscles and transmit the data to the patient monitor 16. The patient monitor 16 may receive the data from the recording electrodes and determine whether the neuromuscular junction is blocked, or in other words, the paralytic state of the patient 12. The patient monitor 16 may be configured to determine paralytic state of the patient 12 based on the data from the recording electrodes and display the paralytic state. In an embodiment, the NMT unit 88 may include both the stimulating electrodes and the recording electrodes. The sensor 14 can thus provide both EEG and NMT signals from the same sensor substrate, enabling a robust set of physiological parameters to be assessed to determine the patient's anesthetic and paralytic state.

In other embodiments, the NMT unit 88 may include the pair of stimulating electrodes without the pair of recording electrodes, and the recording is done by the other EEG/EMG electrodes 50. In the illustrated example of FIG. 2B, the sensor 14 may include the first NMT electrode 90A and the second NMT electrode 90B, which may both be stimulating electrodes. The NMT electrodes 90 may be the pair of stimulating electrodes, and the third electrode 50C and the second electrode 50B may be the pair of recording electrodes. The electrodes 50B, 50C thus perform dual functions-contributing measurements to both the EEG and NMT signals. In certain embodiments, the sensor 14 may include an additional recording electrode between the first electrode 50A and the second electrode 50B. For example, the NMT electrodes 90 may stimulate the nerves of the patient 12 and the third electrode 50C and an additional recording electrode may measure the electrical activity.

As illustrated in FIG. 2B, the first NMT electrode 90A may be positioned within the fourth electrode assembly 52D and the second NMT electrode 90B may be positioned within the third electrode assembly 52C. To accommodate the NMT electrodes 90, the third electrode 50C and the fourth electrode 50D may be shifted along the longitudinal axis 46 such that the electrode assembly 52 surrounds both the NMT electrode 90 and the electrode 50. For example, the first NMT electrode 90A and the fourth electrode 50D may be positioned within the center of the fourth electrode assembly 52D and entirely surrounded by the fourth electrode assembly 52D. Similarly, the second NMT electrode 90B may be positioned adjacent to the third electrode 50C along the longitudinal axis 46, within the third assembly 52C. Although the sensor 14 of FIG. 2B includes two NMT electrodes 90, the sensor 14 may include any suitable number of NMT stimulation electrodes 90 within any of the electrode assemblies 52. For example, the first electrode assembly 52A may include two or more NMT electrodes 90, three or more NMT electrodes 90, four or more NMT electrodes 90, and so on.

When the third electrode 50C and the second electrode 50B are used as the recording electrodes, these electrodes 50 may be configured to sense the electrical activity of the muscle and transmit the data (e.g., electromyography (EMG) data) to the monitor 16. In other embodiments, the sensor 14 may include an accelerometer, a piezoelectric element (such as a film), capacitive sensors, or any suitable sensor for measuring and/or recording muscle movement. Additionally or alternatively, the patient monitoring system 10 may include a camera and/or image sensor for generating and/or transmitting image data to the monitor 16. The patient monitor 16 may be configured to use image analysis techniques, light-speckle contrast imaging, machine learning techniques, artificial intelligence techniques, and the like to identify muscle movement of the patient 12 in response to a stimulating pulse from NMT electrodes 90. By integrating the NMT unit 88 and/or the NMT electrodes 90 into the sensor 14, dual monitoring of both NMT and EEG signals can take place with reduced numbers or amounts of cables, connectors, sensor surface area on the skin, and workflow steps.

A sensor 214 according to an aspect of the present disclosure is depicted in FIG. 2C and FIG. 2D. The sensor 214 is a combined NMT and EEG sensor. NMT monitoring is an additional method for monitoring a patient's status during anesthesia, by monitoring the depth of paralysis or muscle relaxation of the patient. NMT monitoring works by applying a stimulating pulse to a nerve and sensing the resulting muscle response. An NMT stimulation pulse is produced and travels to the patient's nerve, and the nerve stimulation causes a contraction in the corresponding muscle. During a medical procedure, a paralytic agent (such as a neuromuscular blocking agent) may be applied to provide muscle relaxation or paralysis. During the procedure, the muscle response to the NMT stimulation pulse indicates the depth of the neuromuscular block. Monitoring this muscle response can thus guide the administration of neuromuscular blocking agents (NMBAs) during anesthesia and indicate return of muscle activity for safe extubation of the patient during recovery.

The sensor 214 is a combined NMT and EEG sensor, providing measurements of both EEG signals from the patient's brain and NMT signals from muscle reactions due to NMT stimulation. Providing both signals from a single sensor expands the quality of monitoring that can be done for the patient during anesthesia, without adding multiple sensors to the patient and multiple monitors and workflows. However it can be difficult to obtain reliable signals from multiple electrodes looking for different signals at the same sites. This is especially the case when obtaining both NMT and EEG signals, as the NMT signal requires an active stimulation step, which can introduce noise into the electrode signals. Aspects of the disclosure herein present solutions in the form of an additional reference electrode for NMT monitoring placed on the opposite-to-stimulation side of the forehead, to act as an improved reference. This is contrary to conventional approaches that intentionally use the center of the forehead as a reference point in order to provide a side-neutral solution. Here, a combined NMT and EEG sensor deploys a reference electrode on the opposite hemisphere from the stimulating electrodes. Additionally, the stimulating electrodes that deliver the NMT stimulation pulses are positioned above and below the EEG temple electrode. This configuration positions the stimulating sensors over the facial nerve (such as the temporal branch of the facial nerve) for NMT stimulation and away from other sensing electrodes used for detecting the patient's EEG signal, as described further below.

The single sensor 214 carries electrodes that are co-used for both EEG and NMT monitoring. In the example of FIGS. 2C and 2D, the sensor 214 includes seven electrodes carried by five electrode assemblies along a sensor substrate 240. The sensor 214 includes a reference electrode 252R which can be separated from the other electrodes by a flexible tether 269. The flexible tether 269 can have an extended length that enables the reference electrode 252R to be placed on the patient on the opposite side of the head from the other electrodes. The reference electrode is positioned at an opposite end of the sensor body from the stimulating electrodes. The NMT signal is measured with respect to this reference electrode 252R, as will be described in further detail below.

The sensor 214 includes a connector 266 which connects to a sensor cable (such as cable 18 of FIG. 1), and a tail 244 between the connector 266 and the electrodes 250, 290. The electrodes of the sensor 214 include four electrodes 250A, 250B, 250C, and 250D and three electrodes 290A, 290B, 290R. The four electrodes 250A-D are utilized to collect the EEG signal. The three electrodes 290A, 290B, 290R are added to the sensor to elicit an NMT response and measure that response, along with the other electrodes. The combined sensor includes one or more dedicated EEG electrodes, one or more dedicated NMT electrodes, and one or more co-used electrodes. The sensor utilizes these electrodes in different subsets and combinations to produce the EEG and NMT signals.

Moving from right to left in FIG. 2C, the sensor 214 includes the reference electrode 290R carried by a reference electrode assembly 252R, which is connected to the rest of the sensor by the tether 269. Next are three electrode assemblies 252A-C. The first electrode assembly 252A carries a first sensing electrode 250A. The second electrode assembly 252B carries a second sensing electrode 250B. The third electrode assembly 252C carries a third sensing electrode 250C. After a separation along the substrate 240, a fourth electrode assembly 252D is provided, which is longer in longitudinal length than the other electrode assemblies. The fourth electrode assembly 252D includes a fourth sensing electrode 250D positioned between two NMT stimulating electrodes 290A, 290B. The two stimulating electrodes 290A, 290B are connected to respective positive and negative charge sources, to produce a stimulating current that passes from one electrode, through the patient's skin, to the other electrode. In use, the electrode assembly 252D is positioned over a nerve of the patient to stimulate the nerve and elicit a muscle contraction.

The first and/or second electrode 250A, 250B are positioned along the stimulated muscle and sense any resulting muscle contraction that follows from the pulse by the stimulating electrodes 290A, 290B. Thus, the sensor 214 utilizes electrodes for multi-disciplinary sensing, using the same electrode (such as 250A and/or 250B) for both EEG sensing and NMT sensing. For NMT sensing, the sensor 214 positions the reference electrode 290R on the opposite hemisphere of the patient's head from the stimulation and the sensing electrodes. The purpose of this reference electrode is to provide a reference signal, which contains significantly lower muscular response signal than the electrodes 250A, 250B. When an NMT stimulation pulse is produced, the differential signal between the electrode 250A or 250B and the electrode 290R is used to detect the NMT signal. When the NMT pulse is not active, the electrode 250A and/or 250B is used along with the electrodes 250C and 250D to detect the patient's EEG.

The electrical stimulation from the stimulation pulse will travel to the nerve but will also travel some distance through the patient's skin and interstitial tissue, causing electrical signals to reach the sensing electrodes without passing through the nerve and muscle. This stimulation pulse itself is an artifact that is not representative of the muscle response and is thus a source of noise. This artifact is measured by the electrodes 250A, 250B and the electrode 290R and subtracted from the measured NMT signal. The sensor 214 integrates this reference electrode 290R into a single sensor along with the other electrodes (290A, 290B, 250A, 250B) but also can be separated by the tether 269 so that the reference electrode 290R can be positioned on the non-stimulated hemisphere. The reference electrode 290R is placed on the opposite hemisphere (the non-stimulated hemisphere) where it can detect the stimulation signal that travels through the skin; however, since the stimulated muscle response is significantly reduced in this hemisphere, the signal detected by the difference (subtraction) between the electrodes 250A or 250B and 290R is proportional to the muscle contraction intensity. Thus the measured signal represents only the muscle response, and not the stimulation pulse itself. The sensor 214 includes dedicated NMT electrodes where appropriate, but also repurposes other EEG sensors where possible, to produce an efficient layout of electrodes that together can measure both EEG and NMT signals.

Optionally, the tether 269 is included to provide additional configurability for placing the sensor 214. The tether 269 can be a flexible substrate that connects the reference electrode 290R to the sensor 214 structurally and electrically (providing conductive traces to the connector 266). This tether can have an extended length, such that the distance between the electrodes 250A and 290R is at least as long as the distance between the electrodes 250A and 250B. By placing 250A in the middle of the forchead, even with a short tether, the electrode 250R will be placed on the opposite hemisphere. Sensors can be made with various lengths of the tether 269, such as a shorter length for pediatric patients and a longer length for adult patients. The tether 269 can be made flexible so that it can curve or bend to move the reference electrode 290R to a desired location on the patient. The tether may be non-adhesive to facilitate bending, unbending, folding, or unfolding without sticking to itself or other components, so that the medical caregiver can easily position or reposition the reference electrode 290R.

An example of measurement scenarios is presented in FIG. 2C. In an example, the NMT signal is measured between the first electrode 250A and the reference electrode 290R, as shown by the NMT bracket. (Alternatively, the NMT signal may be measured between the second electrode 250B and the reference electrode 290R). The EEG signal is measured between the first electrode 250A and the fourth electrode 250D, as shown by the EEG bracket. In this example the second electrode 250B serves as the patient ground electrode for both the EEG and NMT measurements. The third electrode 250C provides EMG artifact reduction for the EEG measurement.

In an example, the NMT sensor utilizes two stimulating electrodes (290A, 290B), two sensing electrodes (250A, 290R), and one ground electrode (250B). The EEG sensor utilizes three sensing electrodes (250A, 250C, 250D) and one ground electrode (250B). The combined sensor includes some electrodes that are dedicated for NMT sensing, some that are dedicated for EEG sensing, and some that are re-used for both NMT and EEG sensing. Both the NMT and EEG sensors use a common ground electrode, but the signals are measured with respect to different reference electrodes.

A bottom view of the sensor 214 is shown in FIG. 2D. Here, the view shows the electrodes (290A, 250D, 290B, 250C, 250B, 250A, and 290R) in their respective electrode assemblies (252D, 252C, 252B, 252A, 252R). Each electrode is recessed within a respective electrode well 254. The electrode well may also be referred to as a gel pocket, and it receives adhesive and/or conductive gel between the recessed electrode and the patient's skin. From right to left, the reference electrode 290R is seated within an electrode well 254R formed in the electrode assembly 252R. The first electrode 250A is seated within an electrode well 254A in the electrode assembly 252A. The second electrode 250B is seated within an electrode well 254B in the electrode assembly 252B. The third electrode 250C is seated within an electrode well 254C in the electrode assembly 252C. The fourth electrode 250D is seated within an electrode well 254D in the electrode assembly 252D, between two additional wells 254E and 254F. The stimulating electrode 290B is seated within the electrode well 254E, and the stimulating electrode 290A is seated within the electrode well 254F. Each electrode assembly includes a perimeter 259 around the respective electrode well, and the perimeter includes an adhesive layer to secure the sensor to the patient. (For clarity, numeral 259 is indicated in FIG. 2D at electrode 250C, but it should be understood that a similar perimeter exists around each other electrode as well.)

FIG. 2E shows an example of the combined NMT and EEG sensor 214 applied to a patient 212. The sensing and stimulating electrodes are placed on the patient's left hemisphere L, and the reference electrode 290R is placed on the opposite side, on the patient's right hemisphere. The stimulating electrodes 290A, 290B and the fourth electrode 250D are positioned at the patient's left temple, such that the electrodes are placed below, above and at the temple, respectively. Moving up from there, the third electrode 250C and the second electrode 250B are positioned over the patient's left eyebrow, and the first electrode 250A is aligned in the center of the patient's head, above the nose. The reference electrode 290R is positioned above the patient's right eyebrow. The tether 269 extends between. In this example, the connector 266 connects to the reference electrode assembly rather than to the fourth electrode assembly, to demonstrate that the connector and the tail can be positioned on either side of the sensor.

The combined NMT and EEG sensor can also be provided in bilateral form, as shown by sensor 314 of FIG. 2F. In this example, the sensor 314 is a bilateral sensor that measures EEG signals from both hemispheres of the patient's brain. The sensor is oriented with the connector 366 in the middle, extending up from the first electrode 350A, with the body of the sensor 314 extending in two branches on either side of the first electrode 350A. To one side, the sensor includes the second electrode 350B, the third electrode 350C-L (left side of the patient), and the fourth electrode 350D-L (left side of the patient). On the other side, the sensor includes the third electrode 350C-R (right side of the patient) and the fourth electrode 350D-R (right side of the patient). The stimulating electrodes 390A, 390B can be positioned on the electrode assembly with the fourth electrode 350D-L and/or the fourth electrode 350D-R. In this bilateral configuration, the reference electrode for the NMT sensing is provided by one of the EEG sensors on the opposite hemisphere, without the need to add a dedicated NMT reference electrode to the sensor. For example, when the stimulating electrodes 290A, 290B are positioned on the patient's left side (along with electrode 350D-L on the right side of the figure), the third electrode 350C-R can act as the reference electrode, as it will be positioned on the opposite hemisphere of the patient from the stimulation. When the stimulating electrodes 290A, 290B are positioned on the patient's right side (with electrode 350D-R, on the left side of the figure), the third electrode 350C-L or the second electrode 350B can act as the reference electrode for the NMT signal. Thus the bilateral sensor 314 integrates EEG and NMT sensing efficiently into multi-purpose electrodes that accomplish sensing for both EEG and NMT signals. The NMT functionality is added to the sensor by adding the stimulating electrodes 290A, 290B at either or both of the fourth electrodes 350D, and then using the other EEG sensing electrodes of the bilateral sensor to measure the NMT signal.

It should be understood that a dedicated NMT reference electrode can be provided on a bilateral sensor, instead of re-using an EEG sensing electrode. For example, in the sensor 314 of FIG. 2F, a dedicated NMT reference electrode can be provided along one of the sensor arms on the opposite hemisphere as the stimulating electrodes 390A, 390B. This may be desired to simplify signal processing to isolate the NMT reference signal from the other electrodes or to minimize repurposing of electrodes when desired.

The benefit of placing the reference electrode opposite the NMT stimulation was demonstrated with data from two different subjects, shown in FIGS. 2G and 2H. In FIG. 2G on the left of the page, an NMT sensor was placed on the subject with both the stimulation and reference electrodes positioned on the same hemisphere. In FIG. 2H on the right of the page, an NMT sensor such as sensor 214 was placed on the subject with the reference electrode on the opposite hemisphere from the stimulation electrodes. NMT stimulation pulses were delivered in a Train Of Four (TOF) pulses to both subjects, meaning that the NMT response was measured from a set of four pulses of electrical stimulation delivered in rapid sequence. The graphs in FIGS. 2G and 2H depict the NMT signal response on the y-axis, and time on the x-axis. The NMT signal response is determined by subtracting the reference signal (measured by the reference electrode) from the measurement signal (measured by the sensing electrode). The pulses appear much more clearly in FIG. 2H as compared to FIG. 2G. In the configuration of FIG. 2H, the reference electrode produced a reference signal that captured the stimulation pulse without interference from muscle response. This resulted in a cleaner NMT signal in FIG. 2H as compared to FIG. 2G.

With the foregoing in mind, FIGS. 3A-3D are a perspective view of the sensor 14 of FIGS. 2A and 2B coupled to the patient 12. In the illustrated examples, the sticker 64 of the sensor 14 is placed in different positions on the forehead of the patient 12, thereby securing the tail component 44 in different positions to reduce interference and/or movement of the tail component 44, the connector 66, and/or the cable 18 during patient monitoring. Thus, the sensor 14 may be configurable via changing a position of the sticker 64.

FIG. 3A is a perspective view of the sensor 14 of FIG. 2A applied to a patient 12 in a first configuration, in accordance with an aspect of the present disclosure. As illustrated, in use, portions of the sensor 14 may adhere to the temples 120, 122 and/or the forchead 124 of the patient 12 along the longitudinal axis 46 of the sensor 14. For example, the sticker 64 may adhere to a first temple 120 of the patient 12, and the sensor 14 extends across the forchead 124 of the patient 12 to a second temple 122 of the patient 12. The sticker 64 may secure the tail section 60 proximate to the forehead of the patient 12, thereby fixing the tail section 60 in place even during movement of the connector 66 and/or the cable 18. Thus the sticker can reduce and/or eliminate movement of the tail section 60 that may result from movement of the connector 66 and/or the cable 18. Additionally, the sticker 64 may secure the connector 66 and/or the cable 18 proximate to the first temple 120 of the patient 12. The sticker 64 may experience pressure caused by movement of the connector 66 and/or the cable 18. Between the sticker 64 and the connector 66, the sensor 14 may include a short additional tail section 61 configured to limit the movement of the connector 66 and reduce pressure exerted onto the sticker 64. In certain embodiments, a size of the sticker 64 may be less than a size of the electrode assemblies 52. In other embodiments, the size of the sticker 64 may be greater than a size of the electrode assemblies 52.

FIG. 3B is a perspective view of the sensor 14 of FIG. 2A applied to a patient 12 in a second configuration, in accordance with an aspect of the present disclosure. As illustrated, the sticker 64 may adhere to the second electrode assembly 52B. The sensor 14 thereby extends from the center of the forehead 124 to the second temple 122 of the patient 12, leaving the temple 120 free and unobstructed. In this way, the sensor 14 may occupy less space (e.g., cover less surface area) on the temples 120, 122 and the forehead 124 of the patient 12 in comparison to the positioning of the sensor 14 in FIG. 3A. That is, the sensor footprint may be reduced, thereby reducing the area occupied by the sensor 14 and increasing the area on the temple 120, 122 and/or the forehead 124 available for placement of additional technology and/or sensor(s).

By adhering an adhesive surface of the sticker 64 to a top surface of the second electrode assembly 52B, the tail section 60 may fold over the first electrode assembly 52A and the connector 66 may be positioned proximate to the third electrode assembly 52C, as shown in FIG. 3B. The tail section 60 may include the slit 62, which may improve flexibility of the tail section 60 and reduce and/or eliminate pressure caused by rotating the tail section 60. Additionally or alternatively, the first electrode assembly 52A may include a knife-cut 123 or other cut-out portion at a joint between the tail section 60 and the first electrode assembly 52A. The knife-cut 123 permits bending of the tail section 60 around the first electrode assembly 52A and counteracts a 180 degree peel of the tail section 60. In particular, the knife-cut 123 relieves or counteracts lift-off force that would otherwise be applied to the first electrode assembly 52A by the folding and twisting of the tail section 60. As such, pressure exerted by the sensor 14 may be reduced, thereby reducing discomfort and/or markings on the patient's skin.

FIG. 3C is a perspective view of the sensor 14 of FIG. 2A applied to a patient 12 in a third configuration, in accordance with an aspect of the present disclosure. As illustrated, the sticker 64 adheres to the forehead of the patient 12 proximate to the second electrode assembly 52B to reduce the sensor footprint in comparison to the positioning of the sensor 14 in FIG. 3A. In particular, the sticker 64 may be positioned above the second electrode assembly 52B along the transverse axis 48 of the sensor 14. The tail section 60 may fold over the first electrode assembly 52A and/or the second electrode assembly 52B to reduce the area occupied by the sensor 14. For example, the sensor 14 may be positioned to extend from the forehead 124 of the patient 12 to the second temple 122 of the patient 12 by placing the sticker 64 adjacent to the second electrode assembly 52B, leaving the first temple unobstructed. Additionally or alternatively, the sticker 64 may be placed adjacent to the bridge 56 between the first electrode assembly 52A and the second electrode assembly 52B, the bridge 56 between the second electrode assembly 52B and the third electrode assembly 52C, or any suitable area on the forehead 124 of the patient 12 to reduce the sensor footprint.

FIG. 3D is a perspective view of the sensor 14 of FIG. 2A applied to a patient 12 in a fourth configuration, in accordance with an aspect of the present disclosure. As illustrated, the sticker 64 may adhere to the first electrode assembly 52A in a rotated position that rotates the tail section 60 by 270 degrees to adjust a position of the connector 66. For example, the sticker 64 may rotate the position of the connector 66 to exit along the transverse axis 48 of the sensor 14 and direct the position of the connector 66 away from the eyes of the patient 12. In this way, the connector 66 and/or the cable 18 may be kept away from the eyes of the patient 12 and/or from interfering with the clinician during the patient monitoring. In addition, resting the connector 66 on the head of the patient 12 may reduce pressure exerted from the connector 66 that may be caused by gravity. That is, the head may support the weight of the connector 66 during the patient monitoring which reduces pressure caused by the connector 66. The foregoing discussion and FIGS. 3A-D illustrate that the sensor 214 can be manipulated into various different positions to achieve a configuration that is desired depending on the particular patient, procedure, and workflow.

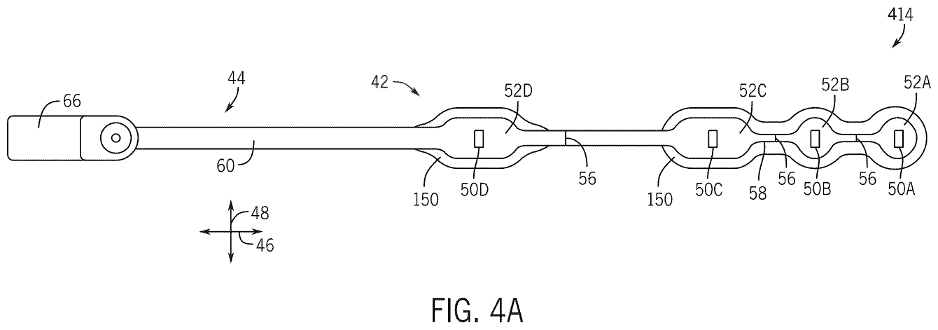

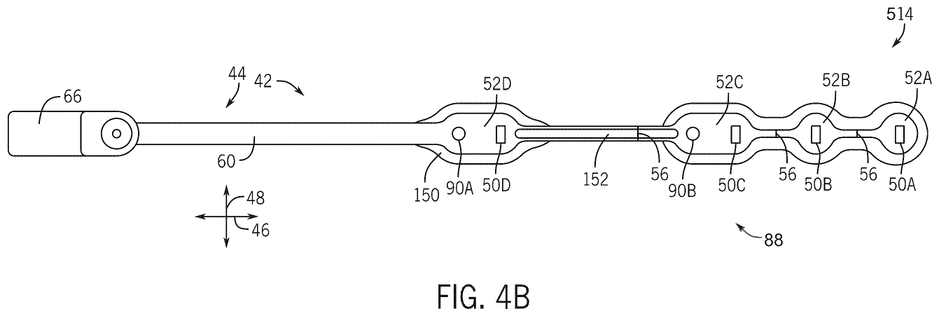

FIG. 4A is a top view of another embodiment of a sensor 414 of the patient monitoring system 10, in accordance with an aspect of the present disclosure. As illustrated, the sensor 414 may include the body component 42 and the tail component 44 positioned along the longitudinal axis 46 of the sensor 414. The body component 42 may include the electrode assemblies 52 surrounding the electrodes 50. The electrode assemblies 52 of FIG. 4A may be narrower than the electrode assemblies 52 of FIG. 2A and the sensor 414 may occupy less area on the forehead of the patient 12 in comparison to the sensor 14 of FIG. 2A. Reducing the size of the sensor 414 may enable the clinician to adhere additional technology and/or additional sensor(s) without manually resizing and/or cutting the sensor 414, thereby simplifying the workflow, reducing setup time, and/or improving operating efficiency. Additionally or alternatively, reducing the footprint of the sensor 414 may enable the sensor 414 to fit on smaller foreheads.

The body component 42 may also include a transparent layer 150 that adheres the sensor 414 to the patient while also aiding the clinician in assessing a condition of the patient's skin below the sensor. The transparent layer 150 may be integrated (e.g., at least partially integrated) with the electrode assembly 52 or surround the electrode assembly 52. The transparent layer 150 may be see-through such that the clinician may view the patient's skin underneath the transparent layer 150. As such, the clinician may assess conditions of the patient's skin, such as developing pressure sores, developing markings, and/or sweating, by viewing portions of the patient's skin though the transparent layer 150. Additionally or alternatively, the transparent layer 150 may include an adhesive (e.g., adhesive layer, gel) configured to adhere to the patient's skin, thereby improving adhesion of the sensor 414. In certain instances, the clinician may adhere additional technologies and/or sensor(s) to the temple 120, 122 and/or the forehead 124 of the patient 12 by overlapping the additional technologies and/or sensor(s) with the transparent layer 150 of the sensor 414. For example, an oximetry sensor with optical emitters and detectors may be positioned over the transparent layer 150 because the transparent layer 150 provides good transmission of the optical signals to the patient's skin. The opportunity to overlap sensors can save space on the patient's forehead, reduce skin irritation, and improve patient monitoring.

In comparison to the sensor 14 of FIGS. 2A and 2B, the sensor 414 of FIG. 4A may be a “reverse tail” sensor, meaning the tail component 44 couples to the fourth electrode assembly 52D instead of the first electrode assembly 52A. Coupling the tail component 44 to the fourth electrode assembly 52D may reduce area occupied by the sensor 414 on the temples 120, 122 and/or forehead of the patient 12. For example, the first electrode assembly 52A may be positioned at a center of the forehead 124 of the patient 12 such that the sensor 414extends along the lontidudinal axis 46 to the second temple 122 of the patient 12. That is, the sensor 414 may not adhere to the first temple 120 of the patient 12. As such, the sensor 414 may occupy less space.

The tail component 44 may include an extended tail section 60 that may be configured to position the connector 66 and/or the cable 18 away from the facial area of the patient 12. For example, the tail section 60 of FIG. 4A may be longer than the tail section 60 of FIG. 2A. The tail section 60 may be positioned between the fourth electrode assembly 52D and the connector 66 and extend along the longitudinal axis 46 of the sensor 414. The fourth electrode assembly 52D may be positioned on the second temple 122 of the patient 12 and position the tail section 60 proximate to the neck of the patient 12. In this way, the tail section 60 may position the connector 66 and/or the cable 18 away from the facial area of the patient 12, which may reduce interference of the cable 18 during patient monitoring. Additionally or alternatively, the tail section 60 may be long enough to facilitate securing the tail section 60, the connector 66, and/or the cable 18 to another object, such as a hospital bed, a table, or the like. Securing the tail section 60, the connector 66, and/or the cable 18 may reduce movement of the components, thereby reducing pressure exerted onto the fourth electrode assembly 52D and reducing markings to the patient's skin.

FIG. 4B is a top view of another embodiment of the sensor 514 of the patient monitoring system 10, in accordance with an aspect of the present disclosure. The sensor 514 of FIG. 4B is substantially similar to the sensor 414 of FIG. 4A except that the sensor 514 of FIG. 4B includes an NMT unit 88 with a first NMT electrode 90A within the fourth electrode assembly 52D and a second NMT electrode 90B within the third electrode assembly 52C. The sensor 514 also includes a color indicator 152.

Between the third electrode assembly 52C and the fourth electrode assembly 52D, the sensor 514 may include the color indicator 152. That is, the bridge 56 between the third electrode assembly 52C and the fourth electrode assembly 52D may include the color indicator 152 or other visual indicator configured to identify the sensor 514 to the clinician. For example, the sensor 514 type may be associated with the color of the color indicator 152 for the clinician to quickly and/or easily identify the type of sensor 514 during patient monitoring. Different sensor types may have different colors or other visual indicators. That is, the clinician may easily identify a type of the sensor 514 based on the color indicator 152. Still, in another example, each patient 12 may be associated with a different color to avoid confusion. As such, the sensor 514 may improve patient monitoring operations.

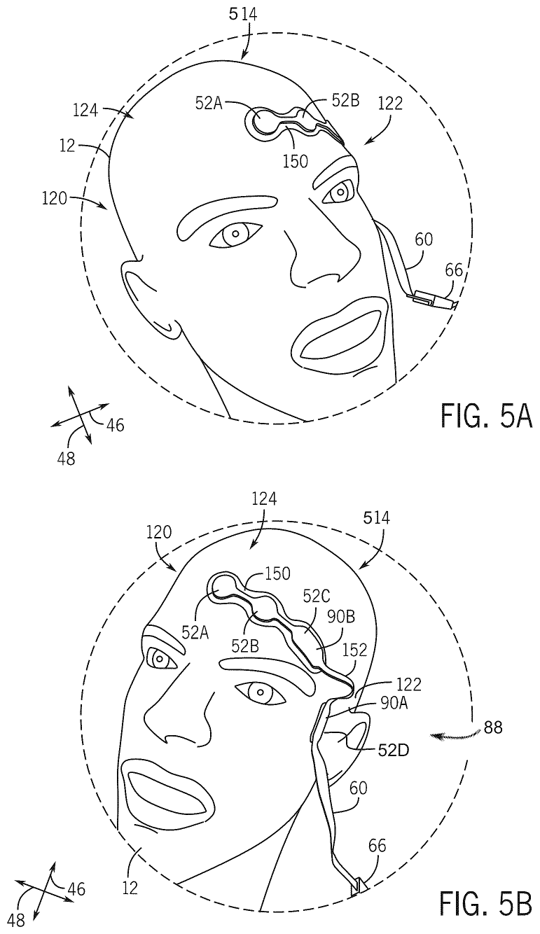

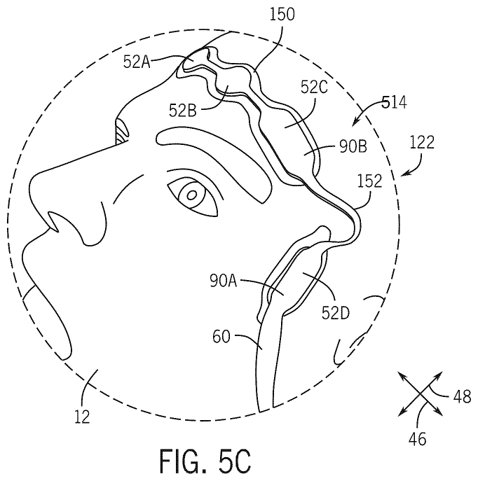

FIG. 5A is a top-down view of the sensor 514 of FIG. 4B applied to a patient 12, in accordance with an aspect of the present disclosure. The sensor 514 may be applied to a center of the forehead 124 of the patient 12 and extend to the second temple 122 of the patient 12. As discussed herein, the sensor 514 of FIG. 4B may include the transparent layer 150 for the clinician to monitor the condition of the patient's skin and/or to layer other technologies and/or sensor(s) to the temple 120, 122 and/or forehead 124 of the patient 12.

As discussed herein, the sensor 514 may include a long tail section 60 coupled to the fourth electrode assembly 52D (visible in FIG. 5B) and configured to position the connector 66 proximate to the neck of the patient 12. As such, the connector 66 may be positioned away from the facial features of the patient 12. Additionally or alternatively, the tail section 60 may be secured to another object to reduce movement of the tail section 60 and/or reduce pressure exerted by the tail section 60 on the patient's skin.

FIG. 5B is a side view of the sensor 514 of FIG. 4B applied to a patient 12, in accordance with an aspect of the present disclosure. As illustrated, the sensor 514 may be configured to be positioned at an angle with respect to the eyebrows of the patient 12. For example, the first electrode assembly 52A may align with the nose the patient 12 along the transverse axis 48, and the second electrode assembly 52B and/or the third electrode assembly 52C may align with the eyebrow of the patient 12. A length of the first electrode assembly 52A, a length of the second electrode assembly 52B, and a length of the third electrode assembly 52C may extend along the longitudinal axis 46 of the sensor 514 and a width of the electrode assemblies 52A-C may extend along the transverse axis 48. The fourth electrode assembly 52D may be positioned proximate to the second temple 122 of the patient 12, such as between the eyebrow and the hairline of the patient 12. Placement of the fourth electrode assembly 52D on the patient may be rotated with respect to the other electrode assemblies 52A-C, such that the fourth electrode 52D extends downward along the patient's face instead of across it.

As discussed herein, the electrodes 50 may monitor physiological parameters of the patient. For example, the first electrode 50A and the fourth electrode 50D may be configured to measure EEG, the third electrode 50C may be configured to measure motion artifacts (for example, twitches or eye blinks) of the patient, and the second electrode 50B may be configured to as a ground.

Additionally or alternatively, the sensor 514 may include the NMT unit 88 configured to measure neuromuscular contractions of the patient 12. The NMT unit 88 may include a first NMT electrode 90A positioned proximate to the second temple 122 of the patient 12 and a second NMT electrode 90B positioned above the eyebrow of the patient 12. The first NMT electrode 90A and the second NMT electrode 90B may be stimulating electrodes configured to stimulate a facial nerve of the patient 12. The third electrode 50C and the fourth electrode 50D may be configured to measure muscle activity caused by stimulating the facial nerve and transmit the muscle activity to the monitor 16. As such, patient monitoring operations may be improved.

FIG. 5C is a close-up side view of the sensor 514 of FIG. 4B applied to a patient 12, in accordance with an aspect of the present disclosure. As illustrated, the third electrode assembly 52C may align with the eyebrow of the patient 12 and the fourth electrode assembly 52D may align with the eyes of the patient 12. Proper placement of the electrode assemblies 52 may properly align the electrodes 50 and/or the NMT electrodes 90 with facial features of the patient 12 to help correctly calculate physiological parameters (e.g., EEG, NMT, paralytic state).

The sensor 514 may include the color indicator 152 between the third electrode assembly 52C and the fourth electrode assembly 52D. Due to the rotated placement of the fourth electrode assembly 52D, the color indicator 152 may extend away from the patient 12 and visually pop out to or grab the attention of the clinician. As such, the sensor 514 may improve ease of use for the clinician. The color indicator 152 may be non-adhesive such that the color indicator is not fully fixed in position relative to the patient but may bend or flex away from the patient between its two fixed or adhered ends (illustrated as adhered electrodes 90A, 90B).

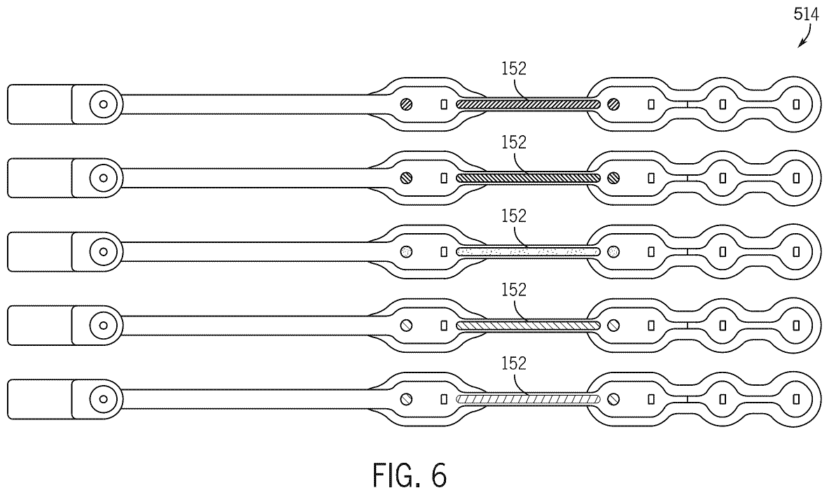

FIG. 6 is a top view of the sensor 514 of FIG. 4B with various color indicators 152, in accordance with aspects of the present disclosure. The colors may provide the clinician a visual indication of the sensor 514. For example, the colors of the color indicator 152 may include gold, silver, red, purple, green, gray, and so on. FIG. 6 depicts these colors in greyscale as different shades of grey.

The color indicator 152 may be configured to provide a visual indicator to the clinician to quickly and/or easily identify the sensor 514. For example, the clinician may associate certain colors with certain sensors and/or their corresponding equipment (e.g., the patient monitor 16). In another example, the clinician may associate certain patients 12 with certain colors.

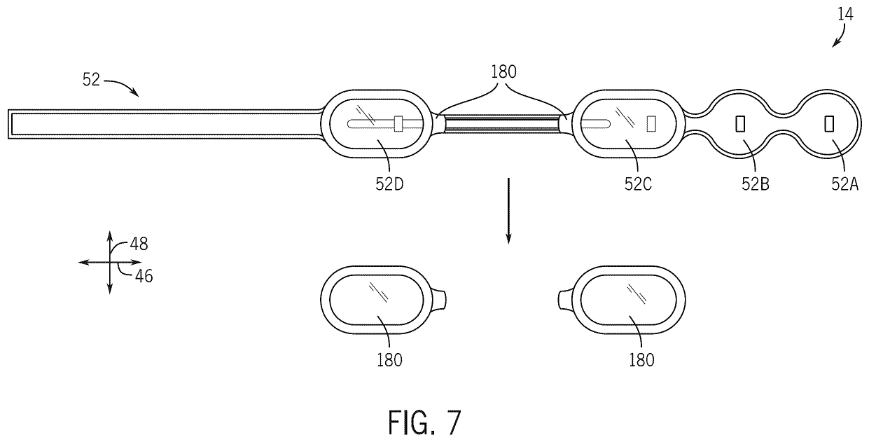

FIG. 7 is a top view of another embodiment of the sensor 14 of the patient monitoring system 10, in accordance with an aspect of the present disclosure. The sensor 14 of FIG. 7 is substantially similar to the sensor 14 of FIG. 4B except that the sensor 14 of FIG. 7 includes patch 180. In certain instances, the clinician may apply a patch to the patient's eyes after induction to keep the eyes closed. To reduce a number of steps performed by the clinician, the sensor 14 may include patches 180 that may be removable from the sensor 14. As illustrated, the patches 180 are seated on a top surface of the third electrode assembly 52C and the fourth electrode assembly 52D. For example, the patches 180 may be shaped similar to the shape of the third electrode assembly 52C and/or the fourth electrode assembly 52D. In other instances, the patches 180 may be seated on other electrodes, such as the first electrode assembly 52A and/or the second electrode assembly 52B. The patches 180 may be any suitable shape and/or size. For example, the patches 180 may be square shaped, triangle shaped, circle shaped, and so on.

The clinician may remove the patches 180 from the sensor 14 before or after adhering the sensor 14 to the temple 120, 122 and/or forehead 124 of the patient 12. As illustrated, the patches 180 may be eye-shaped and configured to cover the eye of the patient 12 during the procedure to keep the eyes closed. The patches 180 may include alternative and/or additional alignment shapes that may be configured to keep the eyes closed, the mouth closed, or the like. As such, the number of steps performed by the clinician prior to patient monitoring may be reduced.

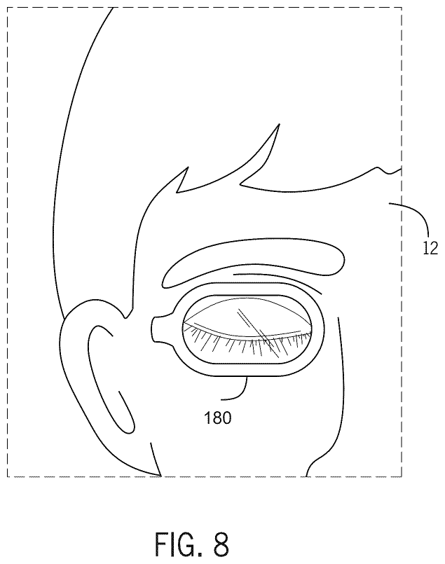

FIG. 8 is a front view of the patches 180 of the sensor 14 of FIG. 7 applied to a patient 12 of the patient monitoring system 10, in accordance with an aspect of the present disclosure. As illustrated, the patches 180 may be positioned to cover the eyes of the patient 12 to keep the eyes closed during patient monitoring. The patches 180 may be removably applied (e.g., removably attached) to the patient 12 by the clinician prior to patient monitoring and may be removed after the patient monitoring. The patches may include an adhesive for this purpose.

FIG. 9 is a top view of another embodiment of the sensor 14 of the patient monitoring system 10, in accordance with an aspect of the present disclosure. In certain instances, the bridges 56 between each electrode assembly 52 may be elastic to improve the ease of use of the sensor 14. For example, the bridges 56 may be made of a flexible and/or elastic material configured to stretch and/or bend. The bridges 56 may extend and/or contract based on the placement of the electrodes 50 and/or the electrode assemblies 52 on the patient 12 based on a size of the patient's head and/or a shape of the patient's head. For example, the distance between each electrode 50 and/or electrode assembly 52 (e.g., the length of each bridge 56) may be based on average head size for a group of adults including males and females. As such, the sensor 14 may not fit as well, for example, for patients with different head shapes, and/or for patients with different head sizes. With elastic bridges 56, the distance between the electrodes 50 and/or the electrode assemblies 52 may be adjusted based on the patient's head. For example, the bridge 56 may extend (e.g., elongate) for proper placement of the electrodes 50 when the patient's head is large and contract (e.g., bend) when the patient's head is small. As such, the electrodes 50 may be properly placed on the patient 12 and configured to facilitate accurate calculations of the physiological parameters.

Additionally or alternatively, the sensor 14 may include alignment features 200 to facilitate positioning of the electrodes 50 and/or the electrode assemblies 52 in a desired position on the temple 120, 122 and/or forehead 124 of the patient 12. The alignment features 200 may illustrate positioning the first electrode 50A at the center of the forehead 124 and vertically aligned with the nose of the patient 12, the second electrode 50B vertically aligned with a first end of the eyebrow of the patient 12, the third electrode 50C vertically aligned with a second end of the eyebrow of the patient 12, and the fourth electrode 50D at the second temple 122 of the patient 12 and horizontally aligned with the eyes of the patient 12. Although not illustrated, the alignment features 200 may also indicate placement for the first NMT electrode 90A and/or the second NMT electrode 90B. As such, the sensor 14 may be configured to facilitate proper placement on the patient 12 to accurately monitor patient parameters.

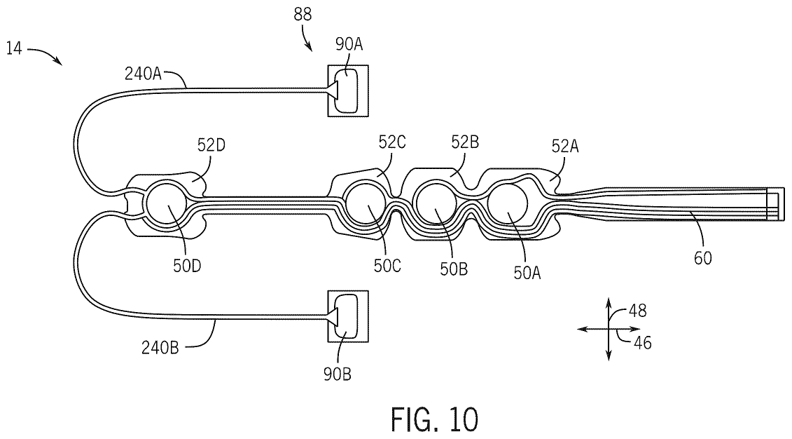

FIG. 10 is a schematic view of another embodiment of the sensor 14 of the patient monitoring system 10, in accordance with an aspect of the present disclosure. As illustrated, the sensor 14 may include an NMT unit 88 with flexible positioning of the first NMT electrode 90A and/or the second NMT electrode 90B. For example, the sensor 14 may include the first NMT electrode 90A extending from the fourth electrode assembly 52D via a first connector 240A and the second NMT electrode 90B extending from the fourth electrode assembly 52D via a second connector 240B. The first connector 240A and/or the second connector 240B (collectively referred to herein as the “connector 240”) may be made from a flexible material, such as the material of the sensor 14.

The connector 240 may be configured to adjust a position and/or orientation of the NMT electrodes 90. For example, a length of the connector 240 may facilitate positioning the NMT electrodes 90 at different positions of the patient 12 and/or the flexibility of the connector 240 may facilitate extending and/or bending of the connector 240 for proper positioning of the NMT electrodes 90. The length of the connector 240 may be longer or shorter than illustrated to facilitate positioning of the NMT electrodes 90 on the patient 12.

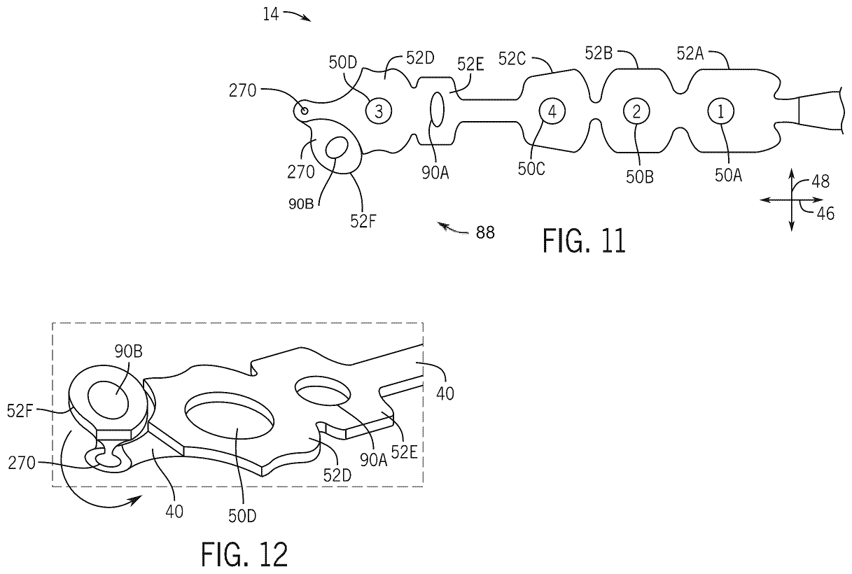

FIG. 11 is a top view of another embodiment of the sensor 14 of the patient monitoring system 10, in accordance with an aspect of the present disclosure. As illustrated, the sensor 14 may include the NMT unit 88 with a first NMT electrode 90A horizontally aligned with the fourth electrode 50D and a second NMT electrode 90B rotatably coupled to the fourth electrode assembly 52D. The sensor 14 may include a fifth electrode assembly 52E that may be horizontally aligned with the fourth electrode assembly 52D. For example, the fifth electrode assembly 52E may be disposed between the third electrode assembly 52C and the fourth electrode assembly 52D. The fifth electrode assembly 52E may be integrated (e.g., at least partially integrated) with the fourth electrode assembly 52D to facilitate proper placement of the first NMT electrode 90A. In this way, the first NMT electrode 90A may align with facial nerves of the patient 12 for stimulation.

The sensor 14 may also include a sixth electrode assembly 52F configured to be rotatably coupled to the fourth electrode assembly 52D by a hinge component 270. The hinge component 270 may extend from the fourth electrode assembly 52D along the longitudinal axis 46 and be configured to couple to the sixth electrode assembly 52F. The hinge component 270 may facilitate the attachment of the sensor 14 to the first temple 120 or the second temple 122. As illustrated in FIG. 12, the substrate 40 may extend along the longitudinal axis 46 past the fourth electrode assembly 52D and form and/or couple to the hinge component 270. The hinge component 270 may be configured to facilitate rotation of the sixth electrode assembly 52F with respect to the fourth electrode assembly 52D and set a position of the sixth electrode assembly 52F. For example, the sixth electrode assembly 52F may be configured to rotate about the hinge component 270 in a clockwise direction and/or a counterclockwise direction. In another example, the sixth electrode assembly 52F may be configured to rotate about the hinge component 270 by 360 degrees or less, such as 270 degrees or less, 180 degrees or less, and so on. To position the second NMT electrode 90B on the patient 12, the clinician may rotate the sixth electrode assembly 52F via the hinge component 270 and then adhere the second NMT electrode 90B at a desired position. In this way, the sensor 14 may be configured to facilitate proper placement of the second NMT electrode 90B, thereby improving generation and/or collection of patient parameters.

FIG. 12 is a close up view of the embodiment of the hinge component 270 of the sensor 14 of FIG. 11 of the patient monitoring system 10, in accordance with an aspect of the present disclosure. As discussed herein, the hinge component 270 may be integrated with and/or coupled to the substrate 40. The hinge component 270 may rotatably couple the sixth electrode assembly 52F to the substrate 40, and also rotatably couple the second NMT electrode 90B to the substrate 40.

When adhering the sensor 14 to a patient 12, the sixth electrode assembly 52F may be rotated via the hinge component 270 to facilitate proper positioning. For example, the clinician may first adhere the first electrode assembly 52A, the second electrode assembly 52B, the third electrode assembly 52C, the fourth electrode assembly 52D, and/or the fifth electrode assembly 52E to the patient 12 and subsequently adhere the sixth electrode assembly 52F to the patient 12. The clinician may rotate the position of the sixth electrode assembly 52F to the proper position with respect to the patient 12. As illustrated, for example, the sixth electrode assembly 52F may be rotated about the hinge component 270 in a clockwise direction to be disposed in the proper position. As such, the positioning of the sensor 14 may be properly aligned with the facial features of the patient 12 and facilitate accurate calculations of the physiological parameters.

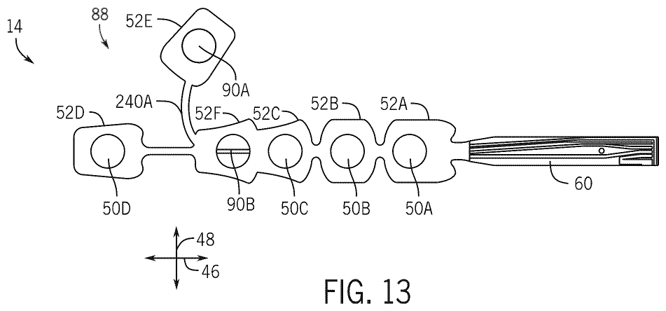

FIG. 13 is a bottom view of another embodiment of the sensor 14 of the patient monitoring system 10, in accordance with an aspect of the present disclosure. The sensor 14 may include the NMT unit 88 configured to stimulate facial nerves of the patient 12 and record muscular activity. As illustrated, the first NMT electrode 90A may be rotatably coupled to the sensor 14 by a first connector 240A and the second NMT electrode 90B may be aligned with the electrodes 50 along the longitudinal axis 46 of the sensor 14. For example, the second NMT electrode 90B may be positioned within a sixth electrode assembly 52F that may be aligned with the first electrode assembly 52A, the second electrode assembly 52B, the third electrode assembly 52C, and/or the fourth electrode assembly 52D along the longitudinal axis 46. The sixth electrode assembly 52F may be positioned between the third electrode assembly 52C and the fourth electrode assembly 52D. In certain instances, the sixth electrode assembly 52F may be integrated (e.g., at least partially integrated) with the third electrode assembly 52C to facilitate proper placement of the second NMT electrode 90B. In other instances, the sixth electrode assembly 52F may not be integrated with any of the electrode assemblies 52 and may be disposed between the third electrode assembly 52C and the fourth electrode assembly 52D.

The first NMT electrode 90A may be disposed within the fifth electrode assembly 52E and may be rotatably coupled to the sixth electrode assembly 52F via the first connector 240A. As discussed herein, the first connector 240A may be made from a flexible material to facilitate proper placement of the first NMT electrode 90A. For example, the first NMT electrode 90A may be positioned at an angle with respect to the second NMT electrode 90B. As such, the first NMT electrode 90A may be properly positioned on the patient 12 for patient monitoring.

The sensor 14 may include two stimulating electrodes of the NMT unit 88 that extend from the fourth electrode assembly 52D. The stimulating electrodes of the NMT may deliver an electric pulse to the tissue of the patient 12. For example, the stimulating electrodes may adhere to a portion of the patient's face and deliver the electric pulse to facial muscles of the patient. The muscle activity may be recorded by the electrodes 50 and transmitted to the monitor 16. The monitor 16 may determine a paralytic state of the patient 12 based on the muscle activity.

FIG. 14 is a block diagram of the patient monitoring system 10, in accordance with an aspect of the present disclosure. The patient monitoring system 10 may include the sensor 14 adhered to a patient for patient monitoring and communicatively coupled to the monitor 16. The monitor 16 may include processing circuitry 280, control circuitry 282, a user interface 284, and/or a memory 286. The user interface 284 may include a display 288, an input device 290, and/or an output device 292. The monitor 16 may be configured to determine and/or display the physiological parameters of the patient 12 during patient monitoring, such as during a surgical procedure. The display 288 may be configured to display the physiological parameters of the patient, and the input device 290 may be configured to receive an input from the user (e.g., clinician). The input device 290 may include function keys (e.g., keys with varying functions), a power switch, adjustment buttons, an alarm silence button, and so forth. The display 288 may include a monitor, cathode ray tube display, a flat panel display such as a liquid crystal (LCD) display, a plasma display, a light emitting diode (LED) display, and/or any other suitable display. In certain embodiments, the display 288 and the input device 290 may be integrated together as one component. The output device 292 may be any suitable audio device configured to generate and output a sound (e.g., alert, notification, noise).

Processing circuitry 280, as well as other processors, processing circuitry, controllers, control circuitry, and the like, described herein, may include one or more processors. Processing circuitry 280 may include any combination of integrated circuitry, discrete logic circuity, analog circuitry, such as one or more microprocessors, digital signal processors (DSPs), application specific integrated circuits (ASICs), or field-programmable gate arrays (FPGAs). In some examples, processing circuitry 280 may include multiple components, such as any combination of one or more microprocessors, one or more DSPs, one or more ASICs, or one or more FPGAs, as well as other discrete or integrated logic circuitry, and/or analog circuitry.

Control circuitry 282 may be operatively coupled processing circuitry 280. Control circuitry 282 is configured to control an operation of sensor 14, such as the electrodes 50 and/or the NMT electrodes 90. In some examples, control circuitry 282 may be configured to provide timing control signals to coordinate operation of electrodes 50 and/or the NMT electrodes 90. For example, the control circuitry 282 may generate one or more timing control signals configured to turn on and off respective electrodes 50 and/or NMT electrodes 90. For example, the control circuitry 282 may turn on the NMT electrodes 90 to stimulate the facial nerves of the patient and subsequently turn on the electrodes 50 to record the muscle activity. In another example, the control circuitry 282 may turn off the NMT electrodes 90 and turn on the electrodes 50 for EEG monitoring.

Memory 286 may be configured to store, for example, monitored physiological parameter values, raw data from the electrodes 50 and/or the NMT electrodes 90, determined EEG statuses, determined paralytic states, or any combination thereof. The memory 286 may also store program instructions, such as one or more program modules that may executable by the processing circuitry 280 for determining the physiological parameters from sensor data transmitted by the electrode 50 and/or the NMT electrode 90. The memory 286 may also store program instructions that, when executed by processing circuitry 280, cause the processing circuitry 280 to provide the functionality ascribed to it herein. The program instructions may be embodied in software, firmware, and/or RAMware. Memory 286 may include any one or more of volatile, non-volatile, magnetic, optical, or electrical media, such as a random access memory (RAM), read-only memory (ROM), non-volatile RAM (NVRAM), electrically-erasable programmable ROM (EEPROM), flash memory, or any other digital media.

The processing circuitry 280 may be configured to receive sensor data from sensor 14 and determine a sedation state based on the sensor data. In particular, the electrodes 50 of the sensor 14 may measure an EEG signal of the patient 12 and transmit an EEG signal to the processing circuitry 280 for determination of the sedation state. The processing circuitry 280 may be configured to algorithmically calculating the sedation from the EEG signal. The sedation state is a measure of a patient's level of consciousness during general anesthesia such as, for example, the BIS™ bispectral index (from Medtronic). The BIS™ bispectral index value represents a dimensionless number (e.g., ranging from 0, i.e., silence, to 100 i.e., fully awake and alert) output from a multivariate discriminate analysis that quantifies the overall bispectral properties (e.g., frequency, power, and phase) of the EEG signal. The processing circuitry 280 may be configured to transmit the BIS™ bispectral index value or other index, metric, or sedation value to the user interface 284 for display to the clinician.

In certain embodiments, the processing circuitry 280 may be configured to receive sensor data from the sensor 14 and determine a paralytic state and a sedation state. In particular, the NMT electrodes 90 stimulate the patient's nerve and recording electrodes (e.g., electrodes 50A, 50B, 50C) may measure and transmit electrical activity of the patient's muscle (e.g., recorded signals) to the processing circuitry 280. The processing circuitry 280 may analyze the measurement to determine whether a neuromuscular junction is blocked, or in other words, the paralytic state of the patient 12. The processing circuitry 280 may be configured to transmit the paralytic state to the user interface 284 for display to the clinician. Additionally or alternatively, the processing circuitry 280 may determine a sedation index or value based on EEG signals from the electrodes 50 and use the sedation value to set one or more NMT parameters, such as the electrical pulse parameters delivered by the NMT electrodes 90, a timing of the electrical pulse, a timing of recording the muscle activity, and so on. The processing circuitry 280 may transmit the NMT parameters to the NMT unit 88. In response to receiving an indication from the user interface 284 to determine the paralytic state of the patient 12, the processing circuitry 280 may instruct the control circuitry 282 to instruct the NMT electrodes 90 to deliver an electrical pulse (e.g., stimuli) to the patient 12. The processing circuitry 280 may also instruct the electrodes 50 to measure an EMG signal of the patient 12. The processing circuitry 280 may receive the resulting EMG signal from the electrodes 50 and determine the paralytic state of the patient 12 based on the EMG signals. The processing circuitry 280 may instruct the user interface 284 to display the paralytic state for the clinician.

In certain instances, the processing circuitry 280 may include signal processing circuitry configured to perform any suitable analog conditioning of the sensed physiological signals. For example, the sensing circuitry may communicate to the processing circuitry 280 an unaltered (e.g., raw) signal. Processing circuitry 280, e.g., signal processing circuitry, may be configured to modify a raw signal to a usable signal by, for example, filtering (e.g., low pass, high pass, band pass, notch, or any other suitable filtering), amplifying, performing an operation on the received signal (e.g., taking a derivative, averaging), performing any other suitable signal conditioning (e.g., converting a current signal to a voltage signal), or any combination thereof In some examples, the conditioned analog signals may be processed by an analog-to-digital converter of the signal processing circuitry to convert the conditioned analog signals into digital signals. In some examples, signal processing circuitry may operate on the analog or digital form of the signals to separate out different components of the signals. In some examples, signal processing circuitry may perform any suitable digital conditioning of the converted digital signals, such as low pass, high pass, band pass, notch, averaging, or any other suitable filtering, amplifying, performing an operation on the signal, performing any other suitable digital conditioning, or any combination thereof. In some examples, signal processing circuitry may decrease the number of samples in the digital detector signals. In some examples, signal processing circuitry may remove dark or ambient contributions to the received signal. Additionally or alternatively, sensing circuitry may include signal processing circuitry to modify one or more raw signals and communicate to the processing circuitry 280 one or more modified signals.

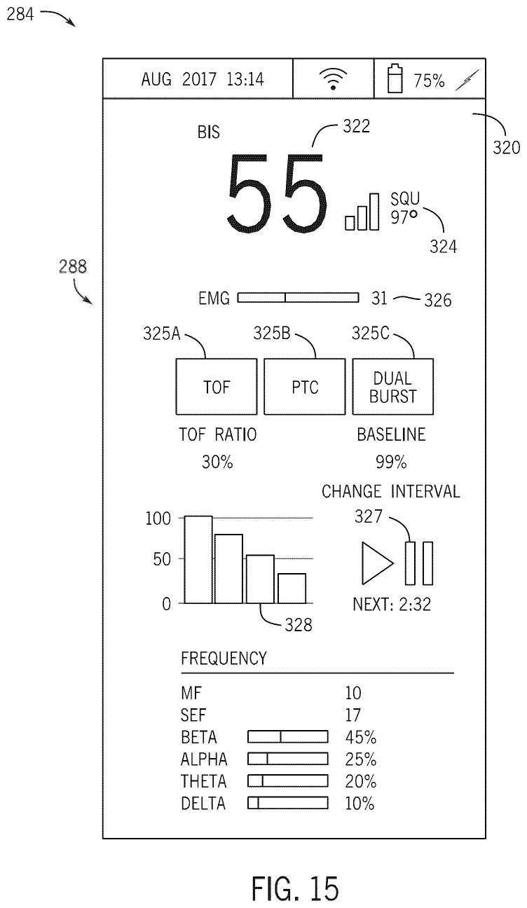

FIG. 15 is a front view of a graphical user interface (GUI) 320 displayed on a user interface 284 of a patient monitor 16 of the patient monitoring system 10, in accordance with an aspect of the present disclosure. The GUI 320 may display physiological parameters of the patient 12, historical trends of physiological parameters, other information about the system (e.g., instructions for placement of the sensor 14 on the patient 12), and/or alarm indications. In particular, the GUI 320 may include a combined EEG and NMT interface displayed on the display 288 of the patient monitor 16.

For example, the GUI 320 may display a sedation value 322, which may be a dimensionless number output from a multivariate discriminate analysis that quantifies the overall properties of the EEG signal. For example, a BIS™ bispectral index value 322 between 40 and 60 may indicate an appropriate level for general anesthesia. The GUI 320 may also display signal quality index (SQU) bar graph 324 (e.g., ranging from 0 to 100) which measures the signal quality of the EEG channel source(s) based on impedance data, artifacts, and other variables. In certain embodiments, the GUI 320 may also display a suppression ratio (SR) which represents the percentage of epochs over a given time period in which EEG signal is considered suppressed, a burst count for the number of EEG bursts per minute, where a “burst” is defined as a short period of EEG activity preceded and followed by periods of inactivity or suppression. In other embodiments, the GUI 320 may be configured to display an EEG waveform. The GUI 320 may also display an electromyograph (EMG) value 326 which may include a spontaneous EMG value of the patient 12.

The GUI 320 may also display buttons 325 with different modes of operation for the NMT electrodes 90. For example, a first button 325A may correspond to a time of flight (TOF) mode, a second button 325B may correspond to a Post-Tetanic Count (PTC) mode, and a third button 325C may correspond to a dual burst mode. The clinician may select a button 325 to select a mode of operation for the NMT electrodes 90. The mode of operation may be manually activated by the clinician or automatically set to trigger in response to lapse of a time period. For example, the NMT electrodes 90 may operate to stimulate the nerve of the patient 12 in response to selection of a button 325. In another example, NMT electrodes 90 may be triggered every 10 minutes, 15 minutes, 20 minutes, or the like in the automatic mode. As illustrated, the GUI 320 may include a timer 327 indicating an amount of time remaining before triggering the NMT electrodes 90.

The clinician may select a button 325 via the GUI 320 to select a mode and/or adjust a GUI displayed on the patient monitor 16. As illustrated, the GUI 320 may display physiological parameters in the TOF mode. For example, the GUI 320 may include a graph 328 indicating a magnitude of each of 4 responses of a TOF measurement. The graph 328 may be adjusted based on the mode of operation. As such, the patient monitor 16 may display the physiological parameters of the patient 12 for the clinician. The clinician may perform one or more operations based on the physiological parameters displayed on the patient monitor 16. For example, the physiological parameter displayed may be a TOF ratio, which may be determined as a ratio of a fourth response to a first response. When less than four responses may be measured (e.g., by the recording electrodes), the TOF ratio may not be calculated and displayed. When four responses may be measured, the TOF ratio may be calculated and displayed. The clinician may determine if a patient 12 may be extubated based on the physiological parameters. For example, the clinician may extubate a patient when the TOF ratio is greater than 0.9. Additionally or alternatively, the patient monitor 16 may display a level of spontaneous EMG. The clinician may consider both the TOF ratio and the level of spontaneous EMG to estimate readiness for extubation.

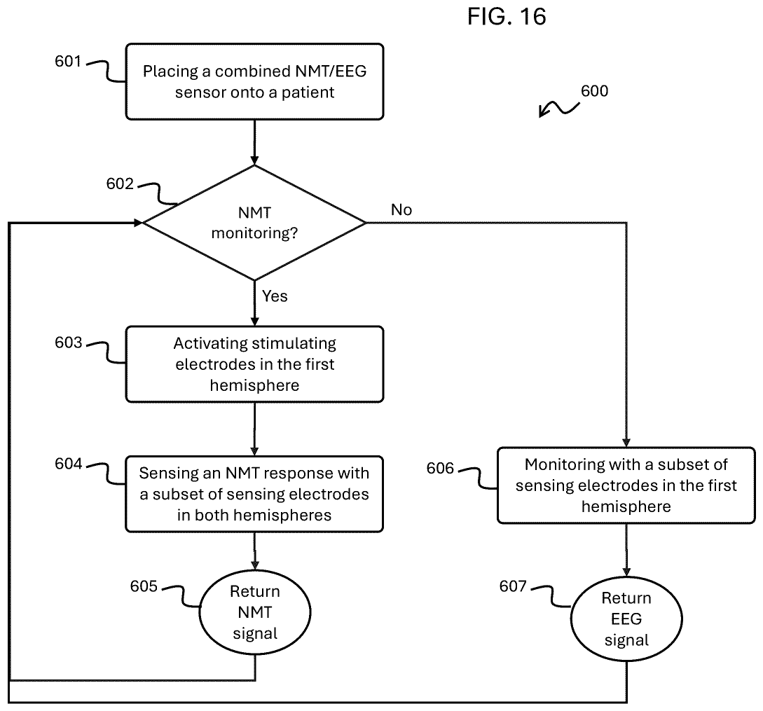

A method 600 of operating a sensor for brain monitoring is depicted in FIG. 16, according to aspects of the disclosure. The method 600 includes placing a combined NMT/EEG sensor onto a patient at 601. In an embodiment, this includes positioning sensing and stimulating electrodes on a first hemisphere of the patient, and positioning a reference sensing electrode on the opposite (non-stimulated) hemisphere of the patient. The method also includes entering an NMT monitoring activity at 602. When NMT monitoring is performed, the method includes activating the stimulating electrodes in the first hemisphere at 603, to deliver a stimulation pulse to the patient, and subsequently sensing an NMT response with a subset of sensing electrodes in both hemispheres, at 604. In an embodiment, this includes sensing a reference signal with the reference electrode in the non-stimulated hemisphere. The method then includes returning an NMT signal at 605. When NMT monitoring is not active or is no longer required following 605, the method includes monitoring with a subset of sensing electrodes in the first hemisphere at 606, and returning an EEG from the patient at 607. If at any point following 607, further NMT monitoring is required, the EEG monitoring is paused and the stimulating electrodes in the first hemisphere are activated at 603 and so on. The EEG and the NMT signals are used to monitor and manage the patient's sedative state and recovery.

While only certain features of the subject matter have been illustrated and described herein, many modifications and changes will occur to those skilled in the art. It is, therefore, to be understood that the appended claims are intended to cover all such modifications and changes as fall within the true spirit of the disclosure. It should be appreciated that any features shown and described with reference to FIGS. 1-16 may be combined in any suitable manner.

In Aspect 1, a sensor includes a flexible substrate having first, second, third, fourth, and fifth electrode assemblies positioned along a longitudinal axis of the substrate; first, second, third, fourth, and fifth sensing electrodes, wherein the first electrode is positioned in a first electrode well of the first electrode assembly, the second electrode is positioned in a second electrode well of the second electrode assembly, the third electrode is positioned in a third electrode well of the third electrode assembly, the fourth electrode is positioned in a fourth electrode well of the fourth electrode assembly, and the fifth electrode is positioned in a fifth electrode well of the fifth electrode assembly; and first and second stimulating electrodes, wherein the fourth sensing electrode is positioned between the first and second stimulating electrodes.

Aspect 2 includes the sensor of Aspect 1, wherein the first, second, third, and fourth electrodes are configured to measure an EEG (electroencephalogram) signal.

Aspect 3 includes the sensor of Aspect 1 or 2, wherein the first, second, and fifth electrodes are configured to measure a neuromuscular transmission (NMT) signal.

Aspect 4 includes the sensor of any preceding Aspect, further comprising: a connector configured to couple the sensor to a monitor; and a non-adhesive tail component extending between the connector and the fourth or fifth electrode assembly; and wherein the fifth electrode assembly is connected to the substrate by a flexible tether.

Aspect 5 includes the sensor of Aspect 4, further comprising a sticker positioned between the tail component and the connector, wherein the sticker is configured to adhere to the patient or the sensor to adjust a position of the tail component, the connector, or both.

Aspect 6 includes the sensor of any preceding Aspect, wherein one of the electrode assemblies is connected to the substrate by a hinge component.

Aspect 7 includes the sensor of Aspect 5, wherein the tail component comprises a slit extending from the sticker to one of the electrode assemblies.

Aspect 8 includes the sensor of any preceding Aspect, comprising a patch removably coupled to at least one of the electrode assemblies.

Aspect 9 includes the sensor of any preceding Aspect, wherein the fifth sensing electrode is positioned at an opposite end of the substrate from the first and second stimulating electrodes.

In Aspect 10, a sensor comprises a first set of electrode assemblies coupled to an adhesive substrate, wherein the first set of electrode assemblies comprises a first sensing electrode and is configured to measure an electroencephalogram (EEG) signal; and a second set of electrode assemblies coupled to the adhesive substrate, wherein the second set of electrode assemblies comprises a pair of stimulating electrodes and is configured to generate a neuromuscular transmission (NMT) signal, wherein the first set of electrode assemblies and the second set of electrode assemblies include at least one common electrode, and wherein the first sensing electrodes is positioned between the pair of stimulating electrodes.

Aspect 11 includes the sensor of Aspect 10, wherein the adhesive substrate comprises a transparent layer.

Aspect 12 includes the sensor of Aspect 10 or 11, further comprising a tail component comprising a sticker.

Aspect 13 includes the sensor of any one of Aspects 10-12, comprising a set of bridges respectively positioned between each electrode assembly of the first set of electrode assemblies, wherein each bridge of the set of bridges comprises a length configured to separate each electrode assembly of the set of electrode assemblies in a longitudinal direction.

Aspect 14 includes the sensor of any one of Aspects 10-13, wherein the common electrode comprises a patient ground electrode.

Aspect 15 includes the sensor of any one of Aspects 10 to 14, wherein the second set of electrodes further comprises a reference electrode positioned at an opposite end of the sensor from the pair of stimulating electrodes.

Aspect 16 includes the sensor of Aspect 15, wherein the reference electrode is coupled to the adhesive substrate by a non-adhesive tether.

Aspect 17 includes the sensor of any one of Aspects 10 to 16, further comprising a third set of electrodes configured to measure an EEG signal on an opposite hemisphere of the patient from the first set of electrodes.

In Aspect 18, a method of operating a sensor for brain monitoring comprises entering a neuromuscular transmission (NMT) monitoring activity executed by a combined NMT/EEG sensor positioned on a patient; activating a pair of stimulating electrodes on the combined NMT/EEG sensor to deliver a stimulation pulse; sensing an NMT response with a first subset of sensing electrodes of the combined NMT/EEG sensor, the first subset including a reference electrode; returning an NMT signal to a patient monitor; alternate to the NMT monitoring activity, monitoring electrical signals of the patient with a second subset of sensing electrodes of the combined NMT/EEG sensor; and returning an EEG signal to the patient monitor.

Aspect 19 includes the sensor of Aspect 18, wherein the first subset of sensing electrodes and the second subset of sensing electrodes include at least one common electrode.

Aspect 20 includes the sensor of Aspect 18, further comprising displaying a sedation state or level of consciousness of the patient derived from the NMT and EEG signals.

The techniques presented and claimed herein are referenced and applied to material objects and concrete examples of a practical nature that demonstrably improve the present technical field and, as such, are not abstract, intangible or purely theoretical. Further, if any claims appended to the end of this specification contain one or more elements designated as “means for (perform)ing (a function) . . . ” or “step for (perform)ing (a function) . . . ”, it is intended that such elements are to be interpreted under 35 U.S.C. 112(f). However, for any claims containing clements designated in any other manner. it is intended that such elements are not to be interpreted under 35 U.S.C. 112(f).

1. A sensor, comprising:
a flexible substrate having first, second, third, fourth, and fifth electrode assemblies positioned along a longitudinal axis of the substrate;
first, second, third, fourth, and fifth sensing electrodes, wherein the first electrode is positioned in a first electrode well of the first electrode assembly, the second electrode is positioned in a second electrode well of the second electrode assembly, the third electrode is positioned in a third electrode well of the third electrode assembly, the fourth electrode is positioned in a fourth electrode well of the fourth electrode assembly, and the fifth electrode is positioned in a fifth electrode well of the fifth electrode assembly; and
first and second stimulating electrodes, wherein the fourth sensing electrode is positioned between the first and second stimulating electrodes.

2. The sensor of claim 1, wherein the first, second, third, and fourth electrodes are configured to measure an EEG (electroencephalogram) signal.

3. The sensor of claim 1, wherein the first, second, and fifth electrodes are configured to measure a neuromuscular transmission (NMT) signal.

4. The sensor of claim 1, further comprising:
a connector configured to couple the sensor to a monitor; and
a non-adhesive tail component extending between the connector and the fourth or fifth electrode assembly; and wherein the fifth electrode assembly is connected to the substrate by a flexible tether.

5. The sensor of claim 4, further comprising a sticker positioned between the tail component and the connector, wherein the sticker is configured to adhere to the patient or the sensor to adjust a position of the tail component, the connector, or both.

6. The sensor of claim 1, wherein one of the electrode assemblies is connected to the substrate by a hinge component.

7. The sensor of claim 5, wherein the tail component comprises a slit extending from the sticker to one of the electrode assemblies.

8. The sensor of claim 1, comprising a patch removably coupled to at least one of the electrode assemblies.

9. The sensor of claim 1, wherein the fifth sensing electrode is positioned at an opposite end of the substrate from the first and second stimulating electrodes.

10. A sensor, comprising:
a first set of electrode assemblies coupled to an adhesive substrate, wherein the first set of electrode assemblies comprises a first sensing electrode and is configured to measure an electroencephalogram (EEG) signal; and
a second set of electrode assemblies coupled to the adhesive substrate, wherein the second set of electrode assemblies comprises a pair of stimulating electrodes and is configured to generate a neuromuscular transmission (NMT) signal,
wherein the first set of electrode assemblies and the second set of electrode assemblies include at least one common electrode, and
wherein the first sensing electrodes is positioned between the pair of stimulating electrodes.

11. The sensor of claim 10, wherein the adhesive substrate comprises a transparent layer.

12. The sensor of claim 10, further comprising a tail component comprising a sticker.

13. The sensor of claim 10, comprising a set of bridges respectively positioned between each electrode assembly of the first set of electrode assemblies, wherein each bridge of the set of bridges comprises a length configured to separate each electrode assembly of the set of electrode assemblies in a longitudinal direction.

14. The sensor of claim 10, wherein the common electrode comprises a patient ground electrode.

15. The sensor of claim 10, wherein the second set of electrodes further comprises a reference electrode positioned at an opposite end of the sensor from the pair of stimulating electrodes.

16. The sensor of claim 15, wherein the reference electrode is coupled to the adhesive substrate by a non-adhesive tether.

17. The sensor of claim 10, further comprising a third set of electrodes configured to measure an EEG signal on an opposite hemisphere of the patient from the first set of electrodes.

18. A method of operating a sensor for brain monitoring, comprising:
entering a neuromuscular transmission (NMT) monitoring activity executed by a combined NMT/EEG sensor positioned on a patient;
activating a pair of stimulating electrodes on the combined NMT/EEG sensor to deliver a stimulation pulse;
sensing an NMT response with a first subset of sensing electrodes of the combined NMT/EEG sensor, the first subset including a reference electrode;
returning an NMT signal to a patient monitor;
alternate to the NMT monitoring activity, monitoring electrical signals of the patient with a second subset of sensing electrodes of the combined NMT/EEG sensor; and
returning an EEG signal to the patient monitor.

19. The method of claim 18, wherein the first subset of sensing electrodes and the second subset of sensing electrodes include at least one common electrode.

20. The method of claim 18. further comprising displaying a sedation state or level of consciousness of the patient derived from the NMT and EEG signals.
